(19) United States
(12) Patent Application Publication
(10) Pub. No.: US 2025/0290117 A1
(43) Pub. Date: Sep. 18, 2025

(54) MOLECULAR PHENOTYPIC ANTIMICROBIAL SUSCEPTIBILITY TESTING

(71) Applicant: Bristol Whiles, Overland Park, KS (US)

(72) Inventor: Bristol Whiles, Overland Park, KS (US)

(21) Appl. No.: 19/062,849

(22) Filed: Feb. 25, 2025

Related U.S. Application Data
(60) Provisional application No. 63/565,420, filed on Mar. 14, 2024.

(51) Int. Cl. C12Q 1/18; C12Q 1/686

(52) U.S. Cl. CPC C12Q 1/18; C12Q 1/686

(57) ABSTRACT

A method for identifying a pathogen includes obtaining a biological specimen. The method may include dividing the specimen into at least a first sample and a second sample. The method may include identifying at least one pathogen in the first sample via genetic analysis. The method may include exposing the second sample to a panel of antimicrobial agents at known concentrations based on the at least one pathogen. The method may include performing a polymerase chain reaction (PCR) assay on the second sample after exposure of the second sample to the panel of the antimicrobial agents, wherein the PCR assay includes oligonucleotides that amplify a nucleic acid sequence for one or more of the identified pathogens. The method may include analyzing a result of the PCR assay.

### CROSS-REFERENCE TO RELATED APPLICATIONS

The present application claims the benefit under 35 U.S.C. § 119 (e) of U.S. Provisional Patent Application Ser. No. 63/565,420, filed Mar. 14, 2024, and entitled MOLECULAR PHENOTYPIC ANTIMICROBIAL SUSCEPTIBILITY TESTING, which is incorporated herein by reference in its entirety.

### TECHNICAL FIELD

The present application is related to the treatment of infections and, more particularly, to the identification of treatment modalities for infections utilizing molecular detection methods for antimicrobial susceptibility.

### BACKGROUND

Infection is a common but typically treatable condition with antimicrobial agents. Pathogen identification is an important first step in the workup, but the pathogen's phenotypic response to antimicrobial treatment, known as antimicrobial susceptibility testing, is important to ensure appropriate treatment for patients with suspected infection. Traditional methods for determining antimicrobial susceptibility testing are slow, taking multiple days to complete before results are available to healthcare providers to aid in clinical decision-making. Newer methods with faster determination of antimicrobial susceptibility are needed.

Therefore, it would be advantageous to provide a system and method that overcomes the shortcomings described above.

### SUMMARY

In one or more embodiments, a method for identifying antimicrobial susceptibility of one or more microorganisms in a biological specimen of a subject is disclosed. In embodiments, the method includes obtaining the biological specimen; dividing the specimen into at least a first sample and a second sample. In embodiments, the method includes identifying at least one pathogen in the first sample via genetic analysis. In embodiments, the method includes exposing the second sample to a panel of antimicrobial agents at known concentrations based on the presence of at least one pathogen. In embodiments, the method includes performing a polymerase chain reaction (PCR) assay on the second sample after exposure of the second sample to the panel of the antimicrobial agents, wherein the PCR assay includes oligonucleotides that amplify a nucleic acid sequence for one or more of the identified pathogens. In embodiments, the method includes analyzing a result of the PCR assay to determine one or more antimicrobial agents that at least one of: inhibit a growth of one or more species of pathogens; reduce a presence of the one or more species of the pathogens; or allow for a growth of the one or more species of the pathogens.

In one or more embodiments, the method includes preparing the first sample for the genetic analysis.

In one or more embodiments, preparing the first sample for the genetic analysis includes: adding a lysis buffer to the first sample; and adding a portion of the lysis buffer to a PCR reaction.

In one or more embodiments, preparing the first sample for the genetic analysis includes: isolating genetic material from the first sample; and adding a portion of the genetic material to a PCR reaction.

In one or more embodiments, the method includes, before performing the PCR assay on the second sample, incubating the second sample in a liquid culture media.

In one or more embodiments of the method the PCR assay is performed based upon an indication that at least of the specimen, the first sample, or the second sample, includes an identified pathogen.

In one or more embodiments, the method includes performing a PCR assay configured to amplify a portion of one or more drug-resistance genes associated with the pathogen from at least one of the biological specimen, the first sample, or the second sample.

In one or more embodiments, the method includes reporting a result of an analysis of the result of the PCR assay to a medical provider.

In one or more embodiments, the method includes administering an effective amount of an antimicrobial agent to the subject based on the analysis of the result of the PCR assay.

In one or more embodiments of the method, identifying the at least one pathogen in the first sample via the genetic analysis includes performing a multiplex PCR reaction.

In one or more embodiments of the method, the multiplex PCR reaction is configured to amplify an identifying DNA sequence from an Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.

In one or more embodiments of the method, the multiplex PCR is further configured to amplify an identifying sequence from Morganella morganii, Ureaplasma urealyticum, and Enterobacter cloacae.

In one or more embodiments of the method, the PCR assay includes semi-quantitative PCR.

In one or more embodiments of the method, the biological specimen includes at least one of urine, blood, saliva, feces, pleural fluid, mucosal samples, or cerebral spinal fluid.

In one or more embodiments of the method, the panel of the antimicrobial agents includes ciprofloxacin and levofloxacin.

A kit is disclosed. In one or more embodiments, the lot includes a polymerase chain reaction (PCR) master mix, wherein the PCR master mix includes: a set of primers for amplifying an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.; a polymerase enzyme; and a PCR buffer solution.

In one or more embodiment of the kit, the set of primers further includes a set of primers for amplifying an identifying DNA sequence from Morganella morganii, Ureaplasma urealyticum, and Enterobacter cloacae.

A system is disclosed. In one or more embodiments, the system includes a polymerase chain reaction (PCR) master mix. In embodiments, the PCR master mix includes: a set of primers for amplifying an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.; a polymerase enzyme; and a PCR buffer solution; and a set of antibiotics configured to treat one or more pathogens identified via amplification of a subject sample via the PCR master mix.

In one or more embodiments of the system, the set of primers further includes primers for amplifying an identifying DNA sequence from Morganella morganii, Ureaplasma urealyticum, and Enterobacter cloacae.

In one or more embodiments of the system, the set of antibiotics includes ciprofloxacin and levofloxacin.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

The numerous advantages of the disclosure may be better understood by those skilled in the art by reference to the accompanying figures.

FIG. 1 illustrates a conceptual view of a method for antimicrobial agent susceptibility testing (AST), in accordance with one or more embodiments of the disclosure.

FIG. 2 illustrates a flow diagram describing a method for antimicrobial agent susceptibility testing (AST), in accordance with one or more embodiments of the disclosure.

### DETAILED DESCRIPTION

Disclosed herein are systems and methods for detecting pathogens in a specimen and performing antimicrobial susceptibility testing (AST) on identified pathogens utilizing a molecular-based platform. The AST includes incubating a sample of the specimen from a subject in media in the presence of an antibiotic agent and then performing genetic analysis on the sample to determine whether the pathogen is sensitive to the antibiotic agent based on the pathogen load. One or more antibiotic agents may then be administered to the subject based on an analysis of the AST result by a healthcare provider.

As used herein, the term “subject” may be used interchangeably with the terms “individual” and “patient” and includes human and non-human subjects. In some embodiments, subjects may be plants, fish, birds, reptiles, or mammals. In some embodiments, the disclosed methods are performed on fungal, bacterial, archaeal, or protozoal cells.

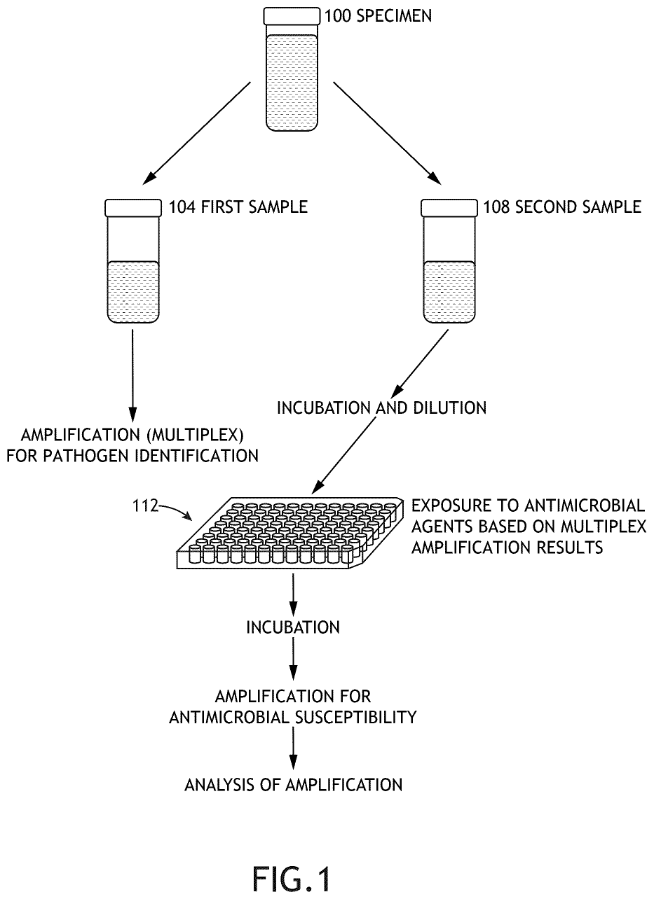

In embodiments, a specimen 100 is obtained from the subject for testing, as shown in FIG. 1. The specimen 100 may include any type of biological material obtained from the subject including, but not limited to, urine, blood, serum, plasma, saliva, cerebral spinal fluid, pleural fluid, milk, lymph, sputum, skin samples, feces, rectal swab, wound samples, vaginal fluid, and semen. For instance, the specimen 100 may be in the form of a fluid or tissue sample including, but not limited, to blood samples, urine samples, plasma samples, saliva samples, pulmonary lavage samples, vaginal secretions, wound lavage, biopsy samples, wound samples, sperm samples, semen samples, prostate fluid samples, cerebrospinal fluid samples, synovial fluid samples, peritoneal fluid samples, pericardial fluid samples, pleural fluid samples, sputum samples, stool samples, mucosal samples, abscess samples, nasal samples, and rectal samples. The specimen 100 may be divided into a first sample 104 (e.g., for pathogen identification), and a second sample 108 (e.g., for AST).

In embodiments, the specimens are collected from subjects using sterile or non-sterile collection processes. For example, the specimens may be collected via methods employed in routine and/or clinical care. For instance, urine samples may be collected by clean catch voided urine or by catheterization. One or more preservatives (e.g., boric acid, EDTA, amines, or other media) may be added to the specimen during the collection process. Once collected, the specimen may be transported to the testing laboratory.

In embodiments, the subject exhibits symptoms of an infection by a pathogen. The pathogen may cause symptoms in the subject that include, but are not limited to fever, discharge, itching, dysuria, hematuria, burning, pain, rash, changes in breathing rate, and increased urinary frequency or urgency. In embodiments, the subject may exhibit no symptoms, and testing may be performed for screening purposes needed in routine and/or clinical care.

In embodiments, the specimen may include one or more infectious agents (e.g., pathogens). The pathogens may be of any kind including but not limited to bacterial pathogens, viral pathogens, fungal pathogens, and protozoan pathogens.

In embodiments, the method includes performing a pathogen identification process for a panel of pathogens on the specimen (e.g., a first sample of the specimen) using genetic sequences for each target of interest via a nucleic acid amplification method including, but not limited to, polymerase chain reaction (PCR). For example, the pathogen identification process may utilize a multiplex amplification (e.g., a multiplex PCR amplification) where specific gene sequences from multiple pathogens are targeted for amplification by sets of gene-specific primers (e.g., oligonucleotides or polynucleotides). In embodiments, the multiplex amplification may include sets of gene-specific primers for more than 10 pathogens, more than 15 pathogens, more than 20 pathogens, more than 30 pathogens, or more than 40 pathogens. For example, the multiplex amplification may include sets of gene-specific primers for 23 distinct pathogens, as shown in table 12A. The multiplex amplification may also include one or more sets of control primers designed to amplify a region of DNA specific to the subject, such as a human subject. For example, the multiplex amplification may include an amplification of one or more of a gene sequence corresponding to the human RNase P gene (e.g., ribonuclease P RNA component H1 (RPPH1)).

In embodiments, the panel of pathogens is formulated based on the specimen source (e.g., urine or blood) and potential infectious organisms. Pathogens included in the panel may include, but are not limited to, Acinetobacter baumannii, Candida albicans, Candida glabrata, Candida parapsilosis, Citrobacter freundii, Enterobacter cloacae, Enterococcus faecalis, Enterococcus faecium, Escherichia coli, Klebsiella/Enterobacter aerogenes, Klebsiella oxytoca, Klebsiella pneumoniae, Morganella morgani, Mycoplasma hominis, Proteus mirabilis, Proteus vulgaris, Providencia stuartii, Pseudomonas aeruginosa, Serratia marcescens, Staphylococcus aureus, Staphylococcus saprophyticus, Streptococcus agalactiae, Ureaplasma urealyticum, Actinotignum schaalii, Aerococcus urinae, Bacteroides fragilis, Candida auris, Chlamydia trachomatis, Citrobacter koseri, Corynebacterium riegelii, Gardnerella vaginalis, Mycoplasma genitalium, Neisseria gonorrhoeae, Pantoea agglomerans, Staphylococcus epidermidis, Staphylococcus saprophyticus, Trichomonas vaginalis.

In embodiments, the method includes exposing the specimen (e.g., a second sample of the specimen) to a panel of antimicrobial agents at known concentrations based on at least one pathogen identified by the pathogen identification process. For example, for the identified pathogen, there may be one or more antimicrobial agents (e.g., antibiotics) known to commonly have activity against that pathogen. The pathogen may then be tested for antimicrobial susceptibility to the antibiotic by incubating the pathogen with the antimicrobial agent for a predetermined time, then testing via a genetic amplification protocol whether or not the pathogen is susceptible or resistant to the antimicrobial agent.

In embodiments, exposing the specimen to the panel of antimicrobial agents includes diluting the second sample into a culture media. The culture media may include any known media that can support the pathogen. Culture media may include, but not be limited to, Muller Hinton Broth (MHB), Tryptic Soy Broth (TSB), Luria Broth (LB), super optimal broth (SOB), and minimum essential medium (MEM).

In embodiments, the second sample is incubated (e.g., 37° C.) until the second sample reaches the predetermined pathogen concentration. For example, the second sample may be incubated for one hour, two hours, four hours, or six or more hours. Once the second sample has reached or exceeded the predetermined pathogen concentration, the second sample may either be used directly or diluted to obtain the specific pathogen concentration of interest. The sample can then either be exposed immediately to test antimicrobial agents or frozen (e.g., at −80° C.) for future use.

In embodiments, dilution of the second sample may include diluting the pathogens in the second sample to a specific pathogen concentration or indicator of pathogen concentration. To dilute the sample to a specific pathogen concentration, an initial determination of the pathogen concentration may be assessed. For example, an Optical Density (OD) of the second sample may be taken (e.g., such as an OD⁶⁰⁰) to determine a predicted pathogen concentration, in another example, a pathogen concentration may be determined via a cell counting method. Once the pathogen concentration is determined, the second sample is diluted to the predetermined concentration (e.g., cells/ml or McFarland Units). For example, the cells may be diluted to 0.5 McFarland units.

In embodiments, the second sample includes more than one pathogen (e.g., pathogen type or pathogen species). For example, for a second sample having more than one pathogen, the second sample may be incubated in a same culture media. In another example, for a second sample having more than one pathogen, the second sample may be incubated in a sample incubation chamber (e.g., tube or plate).

In embodiments, a threshold is used to determine if a second sample will be used for AST. For example, an AST protocol may use a 10,000 cells/ml or 1,000 cells/mL pathogen) concentration as a threshold for entering the second sample into an AST protocol. Other cell concentration thresholds may be used. In embodiments, the identification of the one or more pathogens in the first protocol determined the threshold concentration. For example, a highly virulent bacteria may have a lower pathogen concentration threshold than a less virulent bacteria.

The second samples may be tested for antimicrobial susceptibility in any type of culture container. For example, the second samples may be tested for antimicrobial testing on a plate 112 (e.g., a culture plate containing multiple wells), a culture dish, or a slide). Culture plates included for AST may include but not be limited to 6-well plates, 12-well plates, 24-well plates, 48-well plates, 96-well plates, 384-well plates, and 1536-well plates.

In embodiments, the second sample 108 may be exposed to multiple antimicrobial agents. For example, the second sample may be exposed to one antimicrobial agent, two antimicrobial agents, three antimicrobial agents, four antimicrobial agents, five antimicrobial agents, or six or more antimicrobial agents. In specimens where more than one pathogen is identified, additional plates 112 containing the antimicrobial agents may be used for each pathogen (e.g., particularly those pathogens known to be susceptible to the antimicrobial agent), as different pathogens may likely have different minimum inhibitory concentrations or concentration breakpoints for antibiotic susceptibility determination.

In the embodiments, the second sample 108 may be exposed to each antimicrobial agent at one or more antimicrobial concentrations. For example, the second sample may be exposed to one concentration for a single antimicrobial agent, two concentrations for a single antimicrobial agent, or three or more concentrations for a single antimicrobial agent. In embodiments, the second sample 108 is exposed to two or more antimicrobial agents within the same volume (e.g., a well of a plate 112).

In embodiments, plate 112 includes one or more wells with no antimicrobial agents present. For example, wells may be empty or may contain culture media that can support the pathogen. Culture media may include, but not be limited to, Muller Hinton Broth (MHB), Tryptic Soy Broth (TSB), Luria Broth (LB), super optimal broth (SOB), and minimum essential medium (MEM). For example, the second sample 108 may be inserted into one or more media-only containing wells.

In embodiments, the second sample 108 and/or plate 112 includes one or more pathogens. The pathogens may or may not be individually isolated. For example, the second sample 108 may contain one, two, three, four, or more pathogens together within the same sample.

In embodiments, the plate 112 may be incubated (e.g., 37° C.) to allow for pathogen growth in the presence and absence of antimicrobial agents. For example, the plate may be incubated for one hour, two hours, four hours, six hours, eight hours, twelve hours, 16 hours, or more hours. The plate 112 can either be used directly for AST testing, can be placed in the refrigerator (e.g., at 4° C.) to halt growth for later plate use, or be frozen (e.g., at −80° C.) for future use.

In embodiments, results of the AST are analyzed and generated. For example, the results may be generated based upon the analysis of the Ct (e.g., threshold cycle for a qualitative PCR assay) for one or more pathogens of interest in the control and antimicrobial exposure wells. For instance, the analysis of the Ct may be performed manually (e.g., via visualizing the Ct value from an assay result, or by using a computer software that reports Ct values. The analysis may also be completed via a computer-generated analysis with custom reporting software (using the Ct method or other method for pathogen concentration calculation.

In embodiments, results of the AST are reported (e.g., to a healthcare provider). The AST results may include reporting one or more pathogens as being sensitive, intermediate, resistant, or a combination of those states. For example, an AST report may report a pathogen as being either sensitive or resistant to an antimicrobial agent. In another example, the AST report may report a pathogen as being sensitive, resistant, or having intermediate sensitivity to an antimicrobial agent.

The antimicrobial agents used in the AST protocol may include any antimicrobial agent type including but not limited to aminoglycosides, beta-lactamases, carbabenems, cephalosporins, fluoroquinolones, fosfomycin, linocamides, glycopeptides, lipopeptides, macrolides, monobactams, nitrofurans, oxazolidinones, penicillins, sulfonamides, and tetracyclines.

In some embodiments, the antimicrobial agent is an aminoglycoside. In some embodiments, the antibiotic is selected from amikacin, gentamicin, kanamycin, neomycin, netilmicin, tobramycin, paromomycin, streptomycin, and spectinomycin. In some embodiments, the antimicrobial agent is an ansamycin. In some embodiments, the antibiotic is selected from geldanamycin, herbimycin, and rifaximin. In some embodiments, the antimicrobial agent is a carbapenem. In some embodiments, the antibiotic is selected from ertapenem, doripenem, imipenem, panipenem, biapenem, tebipenem, and meropenem.

In some embodiments, the antimicrobial agent is a cephalosporin. In some embodiments, the antibiotic is selected from cefacetrile, cefadroxil, cephalexin, cefaloglycin, cefalonium, cefaloridine, cefalotin, cefapirin, cefatrizine, cefazaflur, cefazedone, cefazolin, cefradrine, cefroxadine, and ceftezole. In some embodiments, the antimicrobial agent is selected from cefaclor, cefonicid, cefprozil, cefuroxime, cefuzonam, cefmetazole, cefotetan, cefoxitin, loracarbef, cefbuperazone, cefminox, cefoxitin, and cefotiam. In some embodiments, the antimicrobial agent is selected from cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, and latamoxef. In some embodiments, the antimicrobial agent is selected from cefclidine, cefepime, cefluprenam, cefoselis, cefozopran, cefpirome, cefquinome, and flomoxef. In some embodiments, the antibiotic is selected from ceftobiprole, ceftaroline, and ceftolozane.

In some embodiments, the antimicrobial agent is a glycopeptide. In some embodiments, the antimicrobial agent is selected from teicoplanin, vancomycin, telavancin, dalbavancin, ramoplanin, decaplanin, and oritavancin. In some embodiments, the antimicrobial agent is a lincosamide. In some embodiments, the antimicrobial agent is selected from lincomycin, clindamycin, and pirlimycin.

In some embodiments, the antimicrobial agent is daptomycin. In some embodiments, the antibiotic is a macrolide. In some embodiments, the antibiotic is selected from azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin. In some embodiments, the antimicrobial agent is a ketolide. In some embodiments, the antimicrobial agent is selected from telithromycin, cethromycin, and solithromycin. In some embodiments, the antibiotic is a monobactam.

In some embodiments, the antimicrobial agent is selected from aztreonam. In some embodiments, the antibiotic is a nitrofuran. In some embodiments, the antimicrobial agent is selected from diruazone, furazolidone, nifurfoline, nifuroxazide, nifurquinazol, nifurtoinol, nifurzide, nitrofural, and nitrofurantoin. In some embodiments, the antimicrobial agent is an oxazolidinone. In some embodiments, the antimicrobial agent is selected from linezolid, posizolid, tedizolid, radezolid, torezolid, and cycloserine.

In some embodiments, the antibiotic is a penicillin. In some embodiments, the antimicrobial agent is selected from penicillin G, penicillin K, penicillin N, penicillin O, and penicillin V. In some embodiments, the antimicrobial agent is selected from meticillin, nafcillin, oxacillin, cloxacillin, dicloxacillin, and flucoxacillin. In some embodiments, the antibiotic is selected from ampicillin, amoxicillin, pivampicillin, hetacillin, bacampicillin, metampicillin, talampicillin, and epicillin. In some embodiments, the antimicrobial agent is selected from carbenicilin, ticarcillin, and temocillin. In some embodiments, the antimicrobial agent is selected from mezlocillin and piperacillin. In some embodiments, the antimicrobial agent is selected from clavulanic acid, sulbactam, and tazobactam. In some embodiments, the antibiotic is a polypeptide antibiotic. In some embodiments, the antimicrobial agent is selected from bacitracin, colistin, and polymyxin B.

In some embodiments, the antimicrobial agent is a quinolone or fluoroquinolone antibiotic. In some embodiments, the antimicrobial agent is selected from flumequine, oxolinic acid, rosoxacin, cinoxacin, nalidixic acid, and piromidic acid. In some embodiments, the antimicrobial agent is selected from ciprofloxacin, fleroxacin, lomefloxacin, nadifloxacin, norfloxacin, ofloxacin, pefloxacin, rufloxacin, and enoxacin. In some embodiments, the antimicrobial agent is selected from balofloxacin, grepafloxacin, levofloxacin, pazufloxacin, sparfloxacin, temafloxacin, and tosufloxacin. In some embodiments, the antimicrobial agent is selected from clinafloxacin, gatifloxacin, moxifloxacin, sitafloxacin, prulifloxacin, besifloxacin, gemifloxacin, trovafloxacin, delafloxacin, and ozenoxacin.

In some embodiments, the antimicrobial agent is a sulfonamide. In some embodiments, the antimicrobial agent is selected from sulfacetamide, sulfadiazine, sulfadimidine, sulfafurazole, sulfisomidine, sulfadoxine, sulfamethoxazole, sulfamoxole, sulfanitran, sulfadimethoxine, sulfamethoxypyridazine, sulfametoxydiazine, sulfadoxine, sulfametopyrazine, terephtyl, mafenide, sulfanilamide, sulfasalazine, sulfisoxazole, and sulfonamicochrysoidine.

In some embodiments, the antimicrobial agent is a tetracycline. In some embodiments, the antimicrobial agent is selected from tetracycline, chlortetracycline, oxytetracycline, demeclocycline, lymecycline, meclocycline, metacycline, minocycline, and rolitetracycline.

In some embodiments, the antimicrobial agent is selected from clofazimine, dapsone, capreomycin, cycloserine, ethambutol, ethionamide, isoniazid, pyrazinamide, rifampicin, rifabutin, rifapentine, and streptomycin. In another embodiment, the antimicrobial agent is selected from arsphenamide, chloramphenicol, fosfomycin, fusidic acid, metronidazole, mupirocin, platensimycin, quinupristin, dalfopristin, thiamphenicol, tigecycline, and trimethoprim.

In some embodiments, the antimicrobial agent is an azole antifungal. In some embodiments, the antimicrobial agent is selected from bifonazole, butoconazole, clotrimazole, econazole, fenticonazole, isoconazole, ketoconazole, luliconazole, miconazole, omoconazole, oxiconazole, sertaconazole, sulconazole, and tioconazole. In some embodiments, the antimicrobial agent is selected from albaconazole, efinaconazole, epoxiconazole, fluconazole, isavuconazole, itraconazole, posaconazole, propiconazole, ravuconazole, terconazole, and voriconazole.

In some embodiments, the antimicrobial agent is abafungin. In some embodiments, the antimicrobial agent is an echinocandin. In some embodiments, the antimicrobial agent is selected from anidulafungin, caspofungin, and micafungin.

In some embodiments, the antimicrobial agent is a polyene antifungal. In some embodiments, the antimicrobial agent is selected from amphotericin B, candicidin, filipin, hamycin, natamycin, nystatin, and rimocidin. In some embodiments, the antimicrobial agent is selected from griseofulvin, terbinafine, and flucytosine. In embodiments, the antimicrobial agent includes gepotidacin, cefepime-taniborbactam, or pivmecillinam.

In embodiments, the method may or may not include a separate purification step of pathogen genetic material on the second sample 108 after exposure of the second sample to a panel of antimicrobial agents or its control wells prior to performing PCR. For example, extraction of genetic material from pathogen cells may be completed via a sample purification system (e.g., magnetic bead-based separation to isolate and purify DNA). For example, extraction of genetic material from pathogen cells may be completed via cell lysis (e.g., lysis buffer added to each well and incubated in a thermal cycler). For example, this method may involve extraction followed by PCR or can utilize extraction-free direct PCR. In another example, a portion of the sample may be added directly to the PCR reaction (e.g., without lysis buffer).

In embodiments, the method includes performing a PCR on the second sample 108 after exposure of the second sample to the panel of antimicrobial agents (e.g., to determine a number or relative amount of a pathogen genome in the second sample 108). The method may also involve performing PCR on the second sample 108 from one or more control wells containing no antimicrobial agents. For example, after exposing the second sample to the panel of antimicrobial agents (e.g., incubating the diluted second sample with the antimicrobial agent), pathogens from a portion of the second sample are harvested and subjected to nucleic acid amplification. The PCR may be performed via any type of quantitative or semi-quantitative PCR or DNA sequencing protocol including, but not limited to, TaqMan sequencing, next generation sequencing (NGS) and SYBR Green protocols. For specimens that include more than one pathogen, PCR may include multiple sets of primers for amplifying DNA from each pathogen within the second sample, or may include multiple PCR reactions for amplifying DNA from each pathogen separately, or may include a universal primer for amplifying DNA for any of the pathogens (e.g., universal 16S rRNA gene).

In embodiments, the method includes counting cells (e.g., pathogen cells) in the second sample 108 after incubating the second sample 108 in the presence of an antimicrobial agent. For example, the cells may be counted via a cell counter. The number of cells counted would then be compared to a control (e.g., cells not exposed to the antimicrobial agent) to determine whether the pathogen is sensitive or resistant to the antimicrobial agent.

In embodiments, the method includes analyzing a result of the PCR to determine one or more antimicrobial agents to the one or more pathogens are susceptible (e.g., either reducing pathogen number or inhibiting an increase in pathogen number). The analysis may also determine antimicrobial agents that are not effective in either reducing pathogen number or increasing an increase in pathogen number. The analysis may include obtaining threshold cycle (Ct) values, and determining pathogen cell concentrations or relative pathogen cell concentrations based on the Ct values. The analysis may include other types of pathogen cell concentration or relative pathogen cell concentration algorithms.

In embodiments, the method includes administering at least one antimicrobial agent to the subject by a healthcare provider based on the analysis of the PCR (e.g., treating the subject with a therapeutically effective amount of at least one antimicrobial agent). For example, if the specimen includes a single pathogen that is shown via the semi-quantitative PCR to be sensitive to penicillin G at the susceptible breakpoint, a dose of penicillin G may be administered to the patient such that the predicted concentration of the penicillin G at the site of infection (e.g., bladder) would be at an appropriate concentration (e.g., an effective dose that correlates to the concentration of drug given to the pathogen in-vitro). In embodiments, a combination of antimicrobial agents is administered to the subject based on the analysis of the PCR.

In embodiments, the method includes reporting the AST results (e.g., sensitive or resistant, with or without Minimum Inhibitory Concentration values). The AST results may be reported electronically or via printed material. Once reported, information from the AST may be utilized by healthcare providers to assist in selecting a patient-specific treatment regimen for an infection. This information helps in the process of optimal agent selection and prevents overuse or incorrect use of antimicrobials, both of which can occur in cases where antimicrobial susceptibility is not performed for a suspected infection.

In embodiments, one or more genes involved in, or associated with, resistance to antimicrobial agents (e.g., drug-resistant genes) of the pathogen are assessed. For example, a portion of the first or second sample may be used for genetic testing (e.g., PCR) for the existence of one or more drug-resistant genes within the pathogen. For example, the multiplex amplification may include an amplification of one or more of a gene sequence corresponding to a plasmid-mediated fluoroquinolone resistance gene (e.g., qnrA2, AMR Gene Family, quinolone resistance protein). A list of drug-resistant genes is included in Table 9.

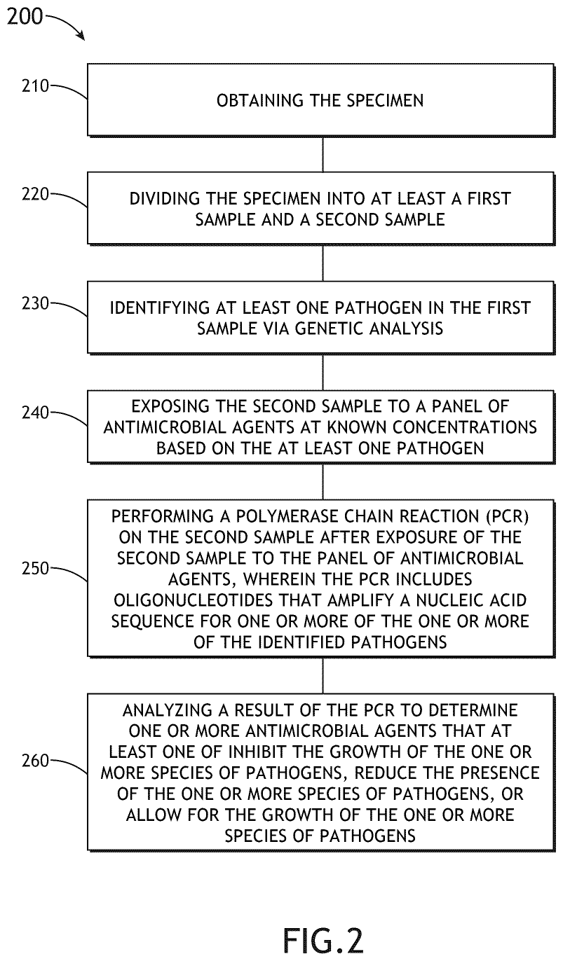

In embodiments, a method 200 for identifying antimicrobial susceptibility of one or more microorganisms in a biological specimen of a subject is disclosed, as shown in FIG. 2, in accordance with one or more embodiments of the disclosure.

In embodiments, the method 200 includes a step 210 of obtaining the specimen 100. In embodiments, the method 200 includes a step 220 of dividing the specimen 100 into at least a first sample 104 and a second sample 108. In embodiments, the method 200 includes a step 230 of identifying at least one pathogen in the first sample 104 via genetic analysis (e.g., via multiplex PCR). In embodiments, the method 200 includes a step 240 of exposing the second sample 108 (e.g., for an incubation period) to a panel of antimicrobial agents at known concentrations based on the at least one pathogen (e.g., the at least one pathogen identified by the multiplex PCR of the first sample 104). In embodiments, the method 200 includes a step 250 of performing a polymerase chain reaction (PCR) on the second sample 108 after exposure and incubation of the second sample to the panel of antimicrobial agents, wherein the PCR includes oligonucleotides (e.g. primers) that amplify a nucleic acid sequence for one or more of the one or more of the identified pathogens. In embodiments, the method 200 includes a step 260 of analyzing a result of the PCR to determine one or more antimicrobial agents that at least one of inhibits the growth of the one or more species of pathogens, reduces the presence of the one or more species of pathogens, or allows for the growth of the one or more species of pathogens. In embodiments, the method 200 further includes a step of administering an antimicrobial agent to the subject based on the analysis of the result of the PCR.

In embodiments, a kit is disclosed. The kit may contain one or more components needed to perform the AST as described herein. For example, the kit may include one or more primer sets as described herein for performing the amplification step to identify one or more pathogens (e.g., for multiplex PCR). In another example, the kit may include one or more primer sets as described herein for performing PCR to determine the antimicrobial susceptibility of the identified one or more pathogens. In another example, the kit may include one or more antimicrobial agents as described herein, one or more control pathogens as described herein, one or more amplification components (e.g., PCR reagents), one or more plates 112, and/or one or more culture media.

In embodiments, the kit includes a polymerase chain reaction (PCR) master mix that includes a set of primers and/or probes for amplifying and/or detecting an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp. The kit may further include a polymerase enzyme and/or a PCR buffer solution. In embodiments, the set of primers and/or probes of the kit may further include primers and/or probes for amplifying and/or detecting an identifying DNA sequence from Morganella morganii, Ureaplasma urealyticum, and Enterobacter cloacae.

In embodiments, a system for performing AST is disclosed. For example, the system may include one or more kits, an incubator, and/or a thermocycler. In embodiments, the system includes a polymerase chain reaction (PCR) master mix that includes a set of primers and/or probes for amplifying and/or detecting an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp. The system may further include a polymerase enzyme and/or a PCR buffer solution. In embodiments, the set of primers and/or probes of the system may further include primers and/or probes for amplifying and/or detecting an identifying DNA sequence from Morganella morganii, Ureaplasma urealyticum, and Enterobacter cloacae.

In embodiments, the system includes a set of antibiotics configured to treat one or more pathogens identified via amplification of a subject sample via the PCR master mix. For instance, the set of antibiotics may include ciprofloxacin and levofloxacin.
a set of antibiotics configured to treat one or more pathogens identified via amplification of a subject sample via the PCR master mix.

In embodiments, the invention herein describes the method for multiple different pathogens but is not limited to the aforementioned pathogens or antimicrobials and/or combinations specifically mentioned.

### EXAMPLES

Herein are examples of the present invention. However, these examples are not limiting examples of the present invention. It is to be understood that these specific examples are not intended to limit the present invention in any way. Equivalents or substitutes are within the scope of the present invention.

### Example 1. Bacterial Organism Detection of Antimicrobial Susceptibility Via Molecular Methods

Detection of Molecular Phenotypic Antimicrobial Susceptibility. Disclosed herein are the methods for the detection of phenotypic antimicrobial susceptibility via molecular methods. The present invention describes how a specimen 100 with a known microbial load is incubated and then exposed to a panel of antimicrobial agents to determine sensitivity (prevention of growth) or resistance (allowance of growth) to the specific antimicrobial agents tested utilizing molecular methods.

Sample Collection. Specimens 100 are collected from patients using sterile collection processes that are employed in routine clinical care. Specimens are transported to the testing facility in boric acid preservative. Specimens 100 are subsequently divided into a first sample 104 and a second sample 108.

Preparation of the Specimens for Testing. The specimens 100 are put through a pathogen identification process using PCR technology (e.g., using the first sample 104). The process for pathogen isolation includes steps of harvesting the pathogen (e.g., via centrifugation), isolating pathogen DNA via a DNA isolation and/or purification kit, and performing PCR on the pathogen DNA via PCR components (e.g., primers, enzymes, buffers) as would be known to the skilled artisan. Simultaneously, an additional aliquot of the same specimen (e.g., the second sample 108) is placed with liquid culture media and incubated for 4-6 hours. This aliquot is used for antimicrobial susceptibility testing if indicated based on the pathogen detection results. After pathogen identification is completed and if more than 10,000 cells/mL of bacteria (e.g., eligible bacteria) are detected in the source specimen, the second sample 108 then enters the antimicrobial susceptibility phase of testing. Specimens 100 with lower pathogen loads could also be utilized in the antibiotic susceptibility phase of testing with or without a longer incubation period.

Preparation of Antimicrobial Susceptibility Testing Plates. Antimicrobial solutions are prepared at known concentrations. The specific antimicrobials and their concentrations are selected based on the pathogen of interest and the described antimicrobial resistance breakpoints for that specimen pathogen and antimicrobial combination. These antimicrobials have been selected based on those commonly utilized by healthcare providers in routine clinical care for the specific type of suspected infection. The number and types of antimicrobials utilized can be customized based on the infection source, pathogen detected, patient-specific factors such as allergies or other health conditions (e.g., chronic kidney disease or concurrent medication use), or healthcare provider preferences such as antimicrobial availability or cost.

The antimicrobial solutions are prepared in liquid culture media (such as Muller Hinton Broth) to achieve the desired antimicrobial concentration and are then stored at −80° C. until use. For a given specimen, each antimicrobial is tested at a single or multiple antimicrobial concentration(s) which is its established breakpoints for sensitivity. The antimicrobial solutions are prepared and plated out for each specific pathogen. This allows ease of use on the day of testing after defrosting the plate 112. Table 1, which illustrates the results of molecular phenotypic antimicrobial susceptibility testing when more than one organism is present in the source specimen, provides an example of an antimicrobial plate 112 prepared for this purpose. Of note, control wells containing only culture media are present on all plates 112. This allows for evaluation of pathogen load when no antimicrobial is present.

Specimen Exposure to Antimicrobial Agents. The 1 mL aliquot of the specimen 100 previously mixed with culture media and incubated is obtained. Using optical density measurements as a guide, the specimen 100 is diluted to 0.5 McFarland. The specimen 100 is then placed into the previously prepared antimicrobial susceptibility testing plate 112. Each plate 112 is prepared for a single specimen 100 for a specified pathogen based on its detection. For example, if the specimen 100 has more than one pathogen detected that needs different antimicrobial concentrations for exposure due to different minimum inhibitory concentration breakpoints for one or more organisms, then more than one antimicrobial exposure plate 112 may be needed. For an additional example, if the specimen 100 has more than one pathogen but their minimum inhibitor concentration breakpoints are the same, then one antimicrobial exposure plate 112 may be utilized. More than one source specimen cannot be tested in a single antimicrobial plate and will need a separate plate 112 for exposure.

Antimicrobial exposure plates are typically placed into an incubator for 12-16 hours. In some scenarios, this incubation process can be completed on a shorter time interval based on the growth parameters of the pathogen being tested (ex: 2-, 4-, 6-, 8-, 10-, or 12-hour incubation).

Nucleic Acid Amplification of Antimicrobial Agent Exposed Pathogens. The antimicrobial exposure plate is removed after the period of incubation. The specimen then undergoes a purification or lysis step. For example, the specimen may undergo a purification step (e.g., magnetic bead-based or other separation protocol to isolate genetic material). In another example, the specimen may undergo a cell lysis step. For example, buffer (e.g., lysis buffer) may be added and incubated in the thermal cycler for preparation for direct PCR. Next, the specimen may undergo a polymerase chain reaction (PCR) testing protocol, whereby a semi-quantitative PCR method is utilized. This may be completed either with or without a specific extraction protocol step. The pathogens can be individually targeted and detected, or a universal genetic sequence can be utilized to detect the antimicrobial susceptibility of the specimen as a whole.

The control wells are utilized as a threshold for determining antimicrobial sensitivity or resistance. Antimicrobial wells with pathogen load detected at equal or higher to the control wells may be reported as Resistant. Antimicrobial wells with pathogen load detected below the control wells (typically >2 cycle thresholds later) may be reported as Sensitive.

Antimicrobial Susceptibility Result Interpretation. The semi-quantitative PCR results for each well in the antimicrobial exposure plate may be compared to control wells to evaluate for resistance (e.g., bacteria multiplied similarly to control wells without antimicrobial agent, despite the exposure to the antimicrobial agent) vs sensitivity (e.g., bacteria did not grow or multiply in the presence of the antimicrobial agent) to the exposed antimicrobial agent. For example, the assay may include the analysis of three control wells to allow for averaging. Analysis using different numbers of control wells (e.g., one control, two controls, three or more control wells) may be used.

The method steps may be repeated for or more, or all, pathogens detected that warrant antimicrobial susceptibility testing. For example: if a specimen had three pathogens, then one, two, or three antimicrobial plates may be needed to ensure exposure at each organism's appropriate antimicrobial breakpoint(s). Regardless of how many antimicrobial plates are utilized, each pathogen is then tested individually by the PCR method for its antimicrobial susceptibility and resistance profile. This method utilized in the test's current iteration ensures that each pathogen is specifically sensitive or resistant to the antimicrobial of interest. This can be completed in a single-plex or multi-plex fashion. Additionally, a universal genetic sequence conserved for all pathogens of interest could be utilized for group assessment of pathogen load in response to antimicrobial exposure to determine overall sensitivity or resistance of the entire specimen. Results are reported as either sensitive or resistant. Intermediate antimicrobial susceptibility or exact minimum inhibitory concentration breakpoints can be determined and reported by a similar process, if desired.

Table 1 illustrates the results of an example antimicrobial agent susceptibility test of the current application versus a traditional culture-based antimicrobial agent susceptibility test for Staphylococcus epidermidis. This specimen underwent extraction or DNA purification prior to PCR. The results of the test of the current application are similar to the results from the traditional test, with the results from the test of the current application being reportable considerably earlier than the test of the traditional test due no pathogen isolation requirement and due to less incubation time being required for the test of the current application.

TABLE 1
Staphylococcus epidermidis molecular phenotypic antimicrobial
susceptibility results vs. traditional culture results

| | | | | | Molecular | | |
|---|---|---|---|---|---|---|---|
| | | | Ct at | Ct at | Antimicrobial | Traditional | |
| | Isolate | | Sensitive | Control | Susceptibility | Culture | |
| Pathogen | Number | Antimicrobial | Breakpoint | (Average) | Result | Result | Match? |
| S. | 1 | TMP/SMX | 10.79 | 10.611 | Resistant | Resistant | Yes |
| Epidermidis | | | | | | | |
| S. | 1 | Cefepime | 11.785 | | Resistant | Resistant | Yes |
| Epidermidis | | | | | | | |
| S. | 1 | Vancomycin | 12.950 | | Sensitive | Sensitive | Yes |
| Epidermidis | | | | | | | |
| S. | 2 | TMP/SMX | 10.503 | 11.861 | Resistant | Resistant | Yes |
| Epidermidis | | | | | | | |
| S. | 2 | Cefepime | 8.669 | | Resistant | Resistant | Yes |
| Epidermidis | | | | | | | |
| S. | 2 | Vancomycin | 18.235 | | Sensitive | Sensitive | Yes |
| Epidermidis | | | | | | | |
| S. | 3 | TMP/SMX | 11.341 | 12.941 | Resistant | Resistant | Yes |
| Epidermidis | | | | | | | |
| S. | 3 | Cefepime | 8.646 | | Resistant | Resistant | Yes |
| Epidermidis | | | | | | | |
| S. | 3 | Vancomycin | 18.173 | | Sensitive | Sensitive | Yes |
| Epidermidis | | | | | | | |
| S. | 4 | TMP/SMX | 10.970 | 11.861 | Resistant | Resistant | Yes |
| Epidermidis | | | | | | | |
| S. | 4 | Cefepime | 8.418 | | Resistant | Resistant | Yes |
| Epidermidis | | | | | | | |
| S. | 4 | Vancomycin | 20.001 | | Sensitive | Sensitive | Yes |
| Epidermidis | | | | | | | |
| S. | 5 | TMP/SMX | 12.620 | 12.941 | Resistant | Resistant | Yes |
| Epidermidis | | | | | | | |
| S. | 5 | Cefepime | 8.474 | | Resistant | Resistant | Yes |
| Epidermidis | | | | | | | |
| S. | 5 | Vancomycin | 8.719 | | Resistant | Sensitive | No |
| Epidermidis | | | | | | | |

Table 2 illustrates the results of an example antimicrobial agent susceptibility test of the current application versus a traditional culture-based antimicrobial agent susceptibility test for Acetobacter baumanii, in accordance with one or more embodiments of the disclosure. This specimen underwent extraction and/or DNA purification prior to PCR. As in Table 1, the results of the test of the current application are similar to the results from the traditional test.

TABLE 2
Acinetobacter baumannii molecular phenotypic antimicrobial
susceptibility results vs. traditional culture results

| | | | | | Molecular | | |
|---|---|---|---|---|---|---|---|
| | | | Ct at | Ct at | Antimicrobial | Traditional | |
| | Isolate | | Sensitive | Control | Susceptibility | Culture | |
| Pathogen | Number | Antimicrobial | Breakpoint | (Average) | Result | Result | Match? |
| A. | 1 | TMP/SMX | 22.888 | 18.811 | Sensitive | Sensitive | Yes |
| baumannii | | | | | | | |
| A. | 1 | Cefepime | 19.944 | | Resistant | Resistant | Yes |
| baumannii | | | | | | | |
| A. | 1 | Ceftriaxone | 16.497 | | Resistant | Resistant | Yes |
| baumannii | | | | | | | |
| A. | 1 | Ciprofloxacin | 16.306 | | Resistant | Resistant | Yes |
| baumannii | | | | | | | |
| A. | 1 | Levofloxacin | 17.268 | | Resistant | Resistant | Yes |
| baumannii | | | | | | | |
| A. | 1 | Meropenem | 18.123 | | Resistant | Resistant | Yes |
| baumannii | | | | | | | |
| A. | 1 | Piperacillin/Tazobactam | 17.127 | | Resistant | Resistant | Yes |
| baumannii | | | | | | | |
| A. | 2 | TMP/SMX | 22.916 | 18.763 | Sensitive | Sensitive | Yes |
| baumannii | | | | | | | |
| A. | 2 | Cefepime | 19.194 | | Resistant | Resistant | Yes |
| baumannii | | | | | | | |
| A. | 2 | Ceftriaxone | 19.075 | | Resistant | Resistant | Yes |
| baumannii | | | | | | | |
| A. | 2 | Ciprofloxacin | 19.471 | | Resistant | Resistant | Yes |
| baumannii | | | | | | | |
| A. | 2 | Levofloxacin | 19.405 | | Resistant | Resistant | Yes |
| baumannii | | | | | | | |
| A | 2 | Meropenem | 19.761 | | Resistant | Resistant | Yes |
| baumannii | | | | | | | |
| A. | 2 | Piperacillin/Tazobactam | 19.691 | | Resistant | Resistant | Yes |
| baumannii | | | | | | | |
| A. | 3 | TMP/SMX | 22.407 | 18.799 | Sensitive | Sensitive | Yes |
| baumannii | | | | | | | |
| A. | 3 | Cefepime | 19.873 | | Sensitive | Sensitive | Yes |
| baumannii | | | | | | | |
| A. | 3 | Ceftriaxone | 19.667 | | Resistant | Resistant | Yes |
| baumannii | | | | | | | |
| A. | 3 | Ciprofloxacin | 17.802 | | Resistant | Resistant | Yes |
| baumannii | | | | | | | |
| A. | 3 | Levofloxacin | 18.253 | | Resistant | Resistant | Yes |
| baumannii | | | | | | | |
| A. | 3 | Meropenem | 18.077 | | Resistant | Resistant | Yes |
| baumannii | | | | | | | |
| A. | 3 | Piperacillin/Tazobactam | 18.647 | | Resistant | Resistant | Yes |
| baumannii | | | | | | | |
| A. | 4 | Ampicillin/Subactam | 19.447 | 18.061 | Sensitive | Sensitive | Yes |
| baumannii | | | | | | | |
| A. | 4 | Gentamicin | 19.380 | | Sensitive | Sensitive | Yes |
| baumannii | | | | | | | |
| A. | 4 | TMP/SMX | 23.297 | | Sensitive | Sensitive | Yes |
| baumannii | | | | | | | |
| A. | 4 | Cefepime | 18.955 | | Resistant | Resistant | Yes |
| baumannii | | | | | | | |
| A. | 4 | Ceftriaxone | 18.774 | | Resistant | Resistant | Yes |
| baumannii | | | | | | | |
| A. | 4 | Ciprofloxacin | 18.101 | | Resistant | Resistant | Yes |
| baumannii | | | | | | | |
| A. | 4 | Levofloxacin | 18.709 | | Resistant | Resistant | Yes |
| baumannii | | | | | | | |
| A. | 4 | Meropenem | 17.553 | | Resistant | Resistant | Yes |
| baumannii | | | | | | | |
| A. | 4 | Piperacillin/Tazobactam | 16.207 | | Resistant | Resistant | Yes |
| baumannii | | | | | | | |
| A. | 5 | Ampicillin/Subactam | 19.682 | 18.191 | Sensitive | Sensitive | Yes |
| baumannii | | | | | | | |
| A. | 5 | Gentamicin | 19.399 | | Sensitive | Sensitive | Yes |
| baumannii | | | | | | | |
| A. | 5 | TMP/SMX | 22.742 | | Sensitive | Sensitive | Yes |
| baumannii | | | | | | | |
| A. | 5 | Cefepime | 18.489 | | Resistant | Resistant | Yes |
| baumannii | | | | | | | |
| A. | 5 | Ceftriaxone | 18.571 | | Resistant | Resistant | Yes |
| baumannii | | | | | | | |
| A. | 5 | Ciprofloxacin | 18.773 | | Resistant | Resistant | Yes |
| baumannii | | | | | | | |
| A. | 5 | Levofloxacin | 19.052 | | Resistant | Resistant | Yes |
| baumannii | | | | | | | |
| A. | 5 | Meropenem | 20.173 | | Resistant | Resistant | Yes |
| baumannii | | | | | | | |
| A. | 5 | Piperacillin/Tazobactam | 17.081 | | Resistant | Resistant | Yes |
| baumannii | | | | | | | |

Table 3 illustrates the results of an example antimicrobial agent susceptibility test of the current application versus a traditional culture-based antimicrobial agent susceptibility test for multiple Escherichia coli isolates, in accordance with one or more embodiments of the disclosure. These specimens underwent extraction or DNA purification prior to PCR. As in Tables 1 and 2, the results of the test of the current application are similar to the results from the traditional test.

TABLE 3
Escherichia coli molecular phenotypic antimicrobial susceptibility results vs. traditional culture results

| | | | | | Molecular | | |
|---|---|---|---|---|---|---|---|
| | | | Ct at | Ct at | Antimicrobial | Traditional | |
| | Isolate | | Sensitive | Control | Susceptibility | Culture | |
| Pathogen | Number | Antimicrobial | Breakpoint | (Average) | Result | Result | Match? |
| E. coli | 1 | TMP/SMX | 32.294 | 21.238 | Sensitive | Sensitive | Yes |
| E. coli | 1 | Nitrofurantoin | 35.690 | | Sensitive | Sensitive | Yes |
| E. coli | 1 | Meropenem | 33.626 | | Sensitive | Sensitive | Yes |
| E. coli | 1 | Gentamicin | 22.585 | | Resistant | Sensitive | No |
| E. coli | 1 | Ceftazidime | 25.605 | | Sensitive | Sensitive | Yes |
| E. coli | 1 | Piperacillin/Tazobactam | 25.572 | | Sensitive | Sensitive | Yes |
| E. coli | 1 | Ampicillin/Sulbactam | 19.827 | | Resistant | Resistant | Yes |
| E. coli | 1 | Cefazolin | 20.925 | | Resistant | Resistant | Yes |
| E. coli | 1 | Ceftriaxone | 19.368 | | Resistant | Resistant | Yes |
| E. coli | 1 | Ciprofloxacin | 20.351 | | Resistant | Resistant | Yes |
| E. coli | 1 | Levofloxacin | 20.768 | | Resistant | Resistant | Yes |
| E. coli | 2 | TMP/SMX | 28.875 | 21.270 | Sensitive | Sensitive | Yes |
| E. coli | 2 | Nitrofurantoin | 30.495 | | Sensitive | Sensitive | Yes |
| E. coli | 2 | Meropenem | 29.525 | | Sensitive | Sensitive | Yes |
| E. coli | 2 | Gentamicin | 24.165 | | Sensitive | Sensitive | Yes |
| E. coli | 2 | Ceftazidime | 22.508 | | Sensitive | Sensitive | Yes |
| E. coli | 2 | Piperacillin/Tazobactam | 22.685 | | Sensitive | Sensitive | Yes |
| E. coli | 2 | Ampicillin/Sulbactam | 21.753 | | Resistant | Resistant | Yes |
| E. coli | 2 | Cefazolin | 19.313 | | Resistant | Resistant | Yes |
| E. coli | 2 | Ceftriaxone | 20.665 | | Resistant | Resistant | Yes |
| E. coli | 2 | Ciprofloxacin | 19.988 | | Resistant | Resistant | Yes |
| E. coli | 2 | Levofloxacin | 19.100 | | Resistant | Resistant | Yes |
| E. coli | 3 | TMP/SMX | 30.605 | 22.0995 | Sensitive | Sensitive | Yes |
| E. coli | 3 | Nitrofurantoin | 21.722 | | Sensitive | Sensitive | Yes |
| E. coli | 3 | Meropenem | 23.588 | | Sensitive | Sensitive | Yes |
| E. coli | 3 | Gentamicin | 25.112 | | Sensitive | Sensitive | Yes |
| E. coli | 3 | Ceftazidime | 22.583 | | Resistant | Sensitive | No |
| E. coli | 3 | Piperacillin/Tazobactam | 20.541 | | Sensitive | Sensitive | Yes |
| E. coli | 3 | Ampicillin/Sulbactam | 20.363 | | Resistant | Resistant | Yes |
| E. coli | 3 | Cefazolin | 20.834 | | Resistant | Resistant | Yes |
| E. coli | 3 | Ceftriaxone | 18.979 | | Resistant | Resistant | Yes |
| E. coli | 3 | Ciprofloxacin | 21.394 | | Resistant | Resistant | Yes |
| E. coli | 3 | Levofloxacin | 29.342 | | Resistant | Resistant | Yes |
| E. coli | 4 | Ciprofloxacin | 17.68 | 17.5733 | Resistant | Resistant | Yes |
| E. coli | 4 | Levofloxacin | 17.88 | | Resistant | Resistant | Yes |
| E. coli | 4 | TMP/SMX | 17.16 | | Resistant | Resistant | Yes |
| E. coli | 4 | Nitrofurantoin | 32.06 | | Sensitive | Sensitive | Yes |
| E. coli | 4 | Ampicillin | 17.20 | | Resistant | Resistant | Yes |
| E. coli | 4 | Cefazolin | 17.23 | | Resistant | Resistant | Yes |
| E. coli | 4 | Cefoxitin | 24.09 | | Sensitive | Sensitive | Yes |
| E. coli | 4 | Cefepime | 17.09 | | Resistant | Resistant | Yes |
| E. coli | 4 | Ceftriaxone | 17.74 | | Resistant | Resistant | Yes |
| E. coli | 4 | Gentamicin | 22.64 | | Sensitive | Sensitive | Yes |
| E. coli | 4 | Piperacillin/Tazobactam | 26.05 | | Sensitive | Sensitive | Yes |
| E. coli | 5 | Ciprofloxacin | 33.02 | 21.0933 | Sensitive | Sensitive | Yes |
| E. coli | 5 | Levofloxacin | 33.85 | | Sensitive | Sensitive | Yes |
| E. coli | 5 | TMP/SMX | 29.12 | | Sensitive | Sensitive | Yes |
| E. coli | 5 | Nitrofurantoin | 33.53 | | Sensitive | Sensitive | Yes |
| E. coli | 5 | Ampicillin | 24.64 | | Sensitive | Sensitive | Yes |
| E. coli | 5 | Cefazolin | 25.36 | | Sensitive | Sensitive | Yes |
| E. coli | 5 | Cefoxitin | 24.72 | | Sensitive | Sensitive | Yes |
| E. coli | 5 | Cefepime | 26.77 | | Sensitive | Sensitive | Yes |
| E. coli | 5 | Ceftriaxone | 25.48 | | Sensitive | Sensitive | Yes |
| E. coli | 5 | Gentamicin | 25.77 | | Sensitive | Sensitive | Yes |
| E. coli | 5 | Piperacillin/Tazobactam | 25.44 | | Sensitive | Sensitive | Yes |

Table 4 illustrates the results of an example antimicrobial agent susceptibility test of the current application for a specimen containing two pathogens of interest, which were incubated together without individual pathogen isolation, in accordance with one or more embodiments of the disclosure.

TABLE 4
Molecular phenotypic antimicrobial susceptibility
results for specimen with two detected pathogens

| | Ct of Controls |
|---|---|
| Pathogen | (Average) |
| E. coli | 15.93 |
| M. morganii | 19.99 |

| | | | Molecular |
|---|---|---|---|
| | | Ct of | Antimicrobial |
| | | Sensitive | Susceptibility |
| Antimicrobial | Pathogen | Breakpoint | Result |
| Ampicillin | E. coli | 15.14 | Resistant |
| | M. morganii | 20.73 | Resistant |
| Ampicillin/Sulbactam | E. coli | 14.58 | Resistant |
| | M. morganii | 22.00 | Sensitive |
| Cefazolin | E. coli | 15.46 | Resistant |
| | M. morganii | 21.60 | Resistant |
| Cefepime | E. coli | 26.80 | Sensitive |
| | M. morganii | 29.72 | Sensitive |
| Cefoxitin | E. coli | 18.30 | Sensitive |
| | M. morganii | 23.01 | Sensitive |
| Ceftriaxone | E. coli | 28.57 | Sensitive |
| | M. morganii | 30.03 | Sensitive |
| Ciprofloxacin | E. coli | 31.81 | Sensitive |
| | M. morganii | 34.71 | Sensitive |
| Fosfomycin | E. coli | 29.24 | Sensitive |
| | M. morganii | 33.43 | Sensitive |
| Gentamicin | E. coli | 17.37 | Resistant |
| | M. morganii | 30.09 | Sensitive |
| Levofloxacin | E. coli | 32.72 | Sensitive |
| | M. morganii | 29.62 | Sensitive |
| Nitrofurantoin | E. coli | 27.36 | Sensitive |
| | M. morganii | 33.37 | Sensitive |
| Meropenem | E. coli | 25.70 | Sensitive |
| | M. morganii | 29.99 | Sensitive |
| Piperacillin/Tazobactam | E. coli | 27.51 | Sensitive |
| | M. morganii | 29.64 | Sensitive |
| Trimethoprim/Sulfamethoxazole | E. coli | 27.67 | Sensitive |
| | M. morganii | 32.93 | Sensitive |
| Tetracycline | E. coli | 14.55 | Resistant |
| | M. morganii | 31.64 | Sensitive |

Table 5 illustrates the results of an example antimicrobial agent susceptibility test of the current application versus a traditional culture-based antimicrobial agent susceptibility test for multiple specimens with different pathogens, in accordance with one or more embodiments of the disclosure. These specimens were tested with direct PCR (e.g., no extraction DNA purification step prior to PCR). As in Tables 1, 2, and 3, the results of the test of the current application are similar to the results from the traditional test.

TABLE 5
Molecular phenotypic antimicrobial susceptibility results with direct PCR vs. traditional culture results

| | | | | | Molecular | | |
|---|---|---|---|---|---|---|---|
| | | | Ct at | Ct at | Antimicrobial | Traditional | |
| | Isolate | | Sensitive | Control | Susceptibility | Culture | |
| Pathogen | Number | Antimicrobial | Breakpoint | (Average) | Result | Result | Match? |
| E. coli | JJ1887 | Ciprofloxacin | 12.215 | 12.832 | Resistant | Resistant | Yes |
| E. coli | JJ1887 | Levofloxacin | 12.562 | | Resistant | Resistant | Yes |
| E. coli | JJ1887 | TMP/SMX | 13.414 | | Resistant | Resistant | Yes |
| E. coli | JJ1887 | Nitrofurantoin | 26.305 | | Sensitive | Sensitive | Yes |
| E. coli | JJ1887 | Amp/Sulbactam | 12.693 | | Resistant | Resistant | Yes |
| E. coli | JJ1887 | Ampicillin | 12.265 | | Resistant | Resistant | Yes |
| E. coli | JJ1887 | Cefazolin | 12.804 | | Resistant | Resistant | Yes |
| E. coli | JJ1887 | Cefepime | 12.796 | | Resistant | Resistant | Yes |
| E. coli | JJ1887 | Ceftriaxone | 12.353 | | Resistant | Resistant | Yes |
| E. coli | JJ1887 | Gentamicin | 12.308 | | Resistant | Resistant | Yes |
| E. coli | JJ1887 | Meropenem | 25.055 | | Sensitive | Sensitive | Yes |
| E. coli | JJ1887 | Pip/Tazo | 23.923 | | Sensitive | Sensitive | Yes |
| S. aureus | NR-46412 | Ciprofloxacin | 18.586 | 18.824 | Resistant | Resistant | Yes |
| | HIP13170 | | | | | | |
| S. aureus | NR-46412 | Levofloxacin | 18.264 | | Resistant | Resistant | Yes |
| | HIP13170 | | | | | | |
| S. aureus | NR-46412 | TMP/SMX | 28.63 | | Sensitive | Sensitive | Yes |
| | HIP13170 | | | | | | |
| S. aureus | NR-46412 | Nitrofurantoin | 31.922 | | Sensitive | Sensitive | Yes |
| | HIP13170 | | | | | | |
| S. aureus | NR-46412 | Gentamicin | 17.179 | | Resistant | Resistant | Yes |
| | HIP13170 | | | | | | |
| S. aureus | NR-46412 | Tetracycline | 18.43 | | Resistant | Resistant | Yes |
| | HIP13170 | | | | | | |
| S. aureus | NR-46412 | Vancomycin | 17.078 | | Resistant | Resistant | Yes |
| | HIP13170 | | | | | | |
| S. aureus | NR-46412 | Cefoxitin | 14.322 | | Resistant | Resistant | Yes |
| | HIP13170 | | | | | | |
| P. aeruginosa | MRSN1938 | Ciprofloxacin | 12.239 | 12.031 | Resistant | Resistant | Yes |
| P. aeruginosa | MRSN1938 | Levofloxacin | 12.853 | | Resistant | Resistant | Yes |
| P. aeruginosa | MRSN1938 | Meropenem | 12.404 | | Resistant | Resistant* | Yes |
| P. aeruginosa | MRSN1938 | Pip/Tazo | 13.375 | | Sensitive | Sensitive | Yes |
| P. aeruginosa | MRSN1938 | Ceftazadime | 24.596 | | Sensitive | Sensitive | Yes |
| P. aeruginosa | MRSN1938 | Cefepime | 13.373 | | Sensitive | Sensitive | Yes |

*Intermediate sensitivity on traditional culture, but only sensitive breakpoint tested so reported as resistant.

Table 6 illustrates the results of an example antimicrobial agent susceptibility test utilizing two different versions of the current application with clinical samples (e.g., extraction DNA purification step prior to PCR vs. direct PCR without a separate DNA purification step). The results of the two applications are similar.

TABLE 6
Molecular phenotypic antimicrobial susceptibility results
with extraction DNA purification vs. direct PCR results

| | | Extraction | | | Direct PCR | | | |
|---|---|---|---|---|---|---|---|---|
| | | | | Molecular | | | Molecular | |
| | | | Ct | Antibiotic | | Ct | Antibiotic | |
| | | | Control | Susceptibility | | control | Susceptibility | |
| Organism | Antimicrobial | Ct | Avg | Result | Ct | avg | Result | Match? |
| E. coli | Ciprofloxacin | 18.022 | 18.112 | Resistant | 12.1 | 12.97 | Resistant | Yes |
| E. coli | Levofloxacin | 17.903 | | Resistant | 12.289 | | Resistant | Yes |
| E. coli | TMP/SMX | 28.072 | | Sensitive | 24.066 | | Sensitive | Yes |
| E. coli | Nitrofurantoin | 29.981 | | Sensitive | 24.883 | | Sensitive | Yes |
| E. coli | Fosfomycin | 28.111 | | Sensitive | 25.481 | | Sensitive | Yes |
| E. coli | Amp/Sulbactam | 17.123 | | Resistant | 13.482 | | Resistant | Yes |
| E. coli | Ampicillin | 18.319 | | Resistant | 12.039 | | Resistant | Yes |
| E. coli | Cefazolin | 26.664 | | Sensitive | 19.983 | | Sensitive | Yes |
| E. coli | Cefoxitin | 26.364 | | Sensitive | 25.006 | | Sensitive | Yes |
| E. coli | Cefepime | 27.275 | | Sensitive | 25.181 | | Sensitive | Yes |
| E. coli | Ceftriaxone | 26.029 | | Sensitive | 24.791 | | Sensitive | Yes |
| E. coli | Gentamicin | 28.366 | | Sensitive | 23.559 | | Sensitive | Yes |
| E. coli | Meropenem | 27.07 | | Sensitive | 24.598 | | Sensitive | Yes |
| E. coli | Pip/Tazo | 26.499 | | Sensitive | 24.816 | | Sensitive | Yes |
| E. coli | Tetracycline | 28.574 | | Sensitive | 26.521 | | Sensitive | Yes |
| P. mirabilis | Ciprofloxacin | 31.142 | 26.064 | Sensitive | 27.139 | 11.801 | Sensitive | Yes |
| P. mirabilis | Levofloxacin | 33.029 | | Sensitive | 25.775 | | Sensitive | Yes |
| P. mirabilis | TMP/SMX | 30.865 | | Sensitive | 23.476 | | Sensitive | Yes |
| P. mirabilis | Nitrofurantoin | 22.903 | | Resistant | 12.013 | | Resistant | Yes |
| P. mirabilis | Fosfomycin | 27.051 | | Sensitive | 26.9 | | Sensitive | Yes |
| P. mirabilis | Amp/Sulbactam | 33.809 | | Sensitive | 28.121 | | Sensitive | Yes |
| P. mirabilis | Ampicillin | 34.177 | | Sensitive | 29.219 | | Sensitive | Yes |
| P. mirabilis | Cefazolin | 22.809 | | Resistant | 21.912 | | Sensitive | No |
| P. mirabilis | Cefoxitin | 29.443 | | Sensitive | 27.055 | | Sensitive | Yes |
| P. mirabilis | Cefepime | 30.77 | | Sensitive | 31.972 | | Sensitive | Yes |
| P. mirabilis | Ceftriaxone | 29.277 | | Sensitive | 22.976 | | Sensitive | Yes |
| P. mirabilis | Gentamicin | 28.953 | | Sensitive | 22.67 | | Sensitive | Yes |
| P. mirabilis | Meropenem | 34.223 | | Sensitive | 32.513 | | Sensitive | Yes |
| P. mirabilis | Pip/Tazo | 28.92 | | Sensitive | 30.658 | | Sensitive | Yes |
| P. mirabilis | Tetracycline | 24.94 | | Resistant | 10.301 | | Resistant | Yes |
| P. mirabilis | Ciprofloxacin | 36.228 | 26.987 | Sensitive | 27.847 | 10.06 | Sensitive | Yes |
| P. mirabilis | Levofloxacin | 33.384 | | Sensitive | 30.018 | | Sensitive | Yes |
| P. mirabilis | TMP/SMX | 33.833 | | Sensitive | 25.628 | | Sensitive | Yes |
| P. mirabilis | Nitrofurantoin | 23.887 | | Resistant | 10.329 | | Resistant | Yes |
| P. mirabilis | Fosfomycin | 31.981 | | Sensitive | 27.935 | | Sensitive | Yes |
| P. mirabilis | Amp/Sulbactam | 36.062 | | Sensitive | 30.953 | | Sensitive | Yes |
| P. mirabilis | Ampicillin | 35.741 | | Sensitive | 30.995 | | Sensitive | Yes |
| P. mirabilis | Cefazolin | 30.359 | | Sensitive | 24.937 | | Sensitive | Yes |
| P. mirabilis | Cefoxitin | 30.603 | | Sensitive | 26.904 | | Sensitive | Yes |
| P. mirabilis | Cefepime | 33.861 | | Sensitive | 29.471 | | Sensitive | Yes |
| P. mirabilis | Ceftriaxone | 32.789 | | Sensitive | 28.394 | | Sensitive | Yes |
| P. mirabilis | Gentamicin | 37.234 | | Sensitive | 30.321 | | Sensitive | Yes |
| P. mirabilis | Meropenem | 35.503 | | Sensitive | 31.805 | | Sensitive | Yes |
| P. mirabilis | Pip/Tazo | 33.895 | | Sensitive | 30.24 | | Sensitive | Yes |
| P. mirabilis | Tetracycline | 23.424 | | Resistant | 11.498 | | Resistant | Yes |
| M. morganii | Ciprofloxacin | Undet | 28.86 | Sensitive | Undet | 12.09 | Sensitive | Yes |
| M. morganii | Levofloxacin | Undet | | Sensitive | Undet | | Sensitive | Yes |
| M. morganii | TMP/SMX | Undet | | Sensitive | 22.916 | | Sensitive | Yes |
| M. morganii | Nitrofurantoin | Undet | | Sensitive | 28.588 | | Sensitive | Yes |
| M. morganii | Fosfomycin | 31.935 | | Sensitive | 18.368 | | Sensitive | Yes |
| M. morganii | Amp/Sulbactam | 37.093 | | Sensitive | 26.942 | | Sensitive | Yes |
| M. morganii | Ampicillin | 25.767 | | Resistant | 10.538 | | Resistant | Yes |
| M. morganii | Cefazolin | 26.596 | | Resistant | 10.461 | | Resistant | Yes |
| M. morganii | Cefoxitin | 37.168 | | Sensitive | 29.041 | | Sensitive | Yes |
| M. morganii | Cefepime | 36.654 | | Sensitive | 28.843 | | Sensitive | Yes |
| M. morganii | Ceftriaxone | 35.749 | | Sensitive | 29.28 | | Sensitive | Yes |
| M. morganii | Gentamicin | Undet | | Sensitive | 32.648 | | Sensitive | Yes |
| M. morganii | Meropenem | 37.898 | | Sensitive | 30.024 | | Sensitive | Yes |
| M. morganii | Pip/Tazo | 36.138 | | Sensitive | 13.986 | | Sensitive | Yes |
| M. morganii | Tetracycline | 26.617 | | Resistant | 9.923 | | Resistant | Yes |
| M. morganii | Ciprofloxacin | 30.662 | 27.053 | Sensitive | 24.534 | 11.677 | Sensitive | Yes |
| M. morganii | Levofloxacin | 37.67 | | Sensitive | 29.614 | | Sensitive | Yes |
| M. morganii | TMP/SMX | 30.225 | | Sensitive | 22.281 | | Sensitive | Yes |
| M. morganii | Nitrofurantoin | Undet | | Sensitive | 29.959 | | Sensitive | Yes |
| M. morganii | Fosfomycin | 37.955 | | Sensitive | 28.123 | | Sensitive | Yes |
| M. morganii | Amp/Sulbactam | Undet | | Sensitive | 32.39 | | Sensitive | Yes |
| M. morganii | Ampicillin | 25.947 | | Resistant | 11.867 | | Resistant | Yes |
| M. morganii | Cefazolin | 25.377 | | Resistant | 12.371 | | Resistant | Yes |
| M. morganii | Cefoxitin | 35.243 | | Sensitive | 29.107 | | Sensitive | Yes |
| M. morganii | Cefepime | 34.238 | | Sensitive | 27.857 | | Sensitive | Yes |
| M. morganii | Ceftriaxone | 36.141 | | Sensitive | 28.625 | | Sensitive | Yes |
| M. morganii | Gentamicin | Undet | | Sensitive | 30.206 | | Sensitive | Yes |
| M. morganii | Meropenem | 36.672 | | Sensitive | 29.381 | | Sensitive | Yes |
| M. morganii | Pip/Tazo | Undet | | Sensitive | 28.975 | | Sensitive | Yes |
| M. morganii | Tetracycline | Undet | | Sensitive | 12.206 | | Resistant | No |
| S. aureus | Ciprofloxacin | 24.118 | 24.32 | Resistant | 16.541 | 20.309 | Resistant | Yes |
| S. aureus | Levofloxacin | 19.106 | | Resistant | 16.32 | | Resistant | Yes |
| S. aureus | TMP/SMX | 27.399 | | Sensitive | 28.965 | | Sensitive | Yes |
| S. aureus | Nitrofurantoin | 30.822 | | Sensitive | 32.477 | | Sensitive | Yes |
| S. aureus | Gentamicin | Undet | | Sensitive | 31.422 | | Sensitive | Yes |
| S. aureus | Tetracycline | 15.029 | | Resistant | 22.653 | | Resistant | Yes |
| S. aureus | Vancomycin | 10.522 | | Resistant | 21.258 | | Resistant | Yes |
| S. aureus | Cefoxitin | 15.037 | | Resistant | 20.586 | | Resistant | Yes |
| E. faecalis | Ciprofloxacin | 35.904 | 22.987 | Sensitive | 32.444 | 22.011 | Sensitive | Yes |
| E. faecalis | Levofloxacin | 37.857 | | Sensitive | 33.342 | | Sensitive | Yes |
| E. faecalis | Ampicillin | 31.697 | | Sensitive | 29.527 | | Sensitive | Yes |
| E. faecalis | Nitrofurantoin | 38.744 | | Sensitive | 36.826 | | Sensitive | Yes |
| E. faecalis | Fosfomycin | 35.358 | | Sensitive | 30.578 | | Sensitive | Yes |
| E. faecalis | Tetracycline | 19.484 | | Resistant | 21.199 | | Resistant | Yes |
| E. faecalis | Vancomycin | 25.491 | | Sensitive | 30.062 | | Sensitive | Yes |
| E. faecalis | Ciprofloxacin | 35.571 | 23.073 | sensitive | 33.973 | 21.027 | sensitive | Yes |
| E. faecalis | Levofloxacin | 38.337 | | sensitive | 34.507 | | sensitive | Yes |
| E. faecalis | Ampicillin | 37.6 | | sensitive | 33.467 | | sensitive | Yes |
| E. faecalis | Nitrofurantoin | 37.449 | | sensitive | 33.684 | | sensitive | Yes |
| E. faecalis | Fosfomycin | 36.898 | | sensitive | 33.882 | | sensitive | Yes |
| E. faecalis | Tetracycline | 23.171 | | Resistant | 20.684 | | Resistant | Yes |
| E. faecalis | Vancomycin | 37.981 | | sensitive | 35.563 | | sensitive | Yes |
| E. faecalis | Ciprofloxacin | 22.597 | 22.144 | Resistant | 21.185 | 20.529 | Resistant | Yes |
| E. faecalis | Levofloxacin | 29.347 | | sensitive | 28.107 | | sensitive | Yes |
| E. faecalis | Ampicillin | 29.684 | | sensitive | 27.555 | | sensitive | Yes |
| E. faecalis | Nitrofurantoin | 32.584 | | sensitive | 32.294 | | sensitive | Yes |
| E. faecalis | Fosfomycin | 20.142 | | Resistant | 19.527 | | Resistant | Yes |
| E. faecalis | Tetracycline | 28.949 | | sensitive | 28.789 | | sensitive | Yes |
| E. faecalis | Vancomycin | 32.184 | | sensitive | 29.81 | | sensitive | Yes |
| E. faecalis | Ciprofloxacin | 31.524 | 20.822 | Sensitive | 28.484 | 20.909 | Sensitive | Yes |
| E. faecalis | Levofloxacin | 32.971 | | Sensitive | 30.072 | | Sensitive | Yes |
| E. faecalis | Ampicillin | 33.01 | | Sensitive | 27.767 | | Sensitive | Yes |
| E. faecalis | Nitrofurantoin | 34.318 | | Sensitive | 32.771 | | Sensitive | Yes |
| E. faecalis | Fosfomycin | 30.598 | | Sensitive | 27.178 | | Sensitive | Yes |
| E. faecalis | Tetracycline | 21.77 | | Resistant | 21.446 | | Resistant | Yes |
| E. faecalis | Vancomycin | 34.122 | | Sensitive | 27.522 | | Sensitive | Yes |
| K. pneumoniae | Ciprofloxacin | 33.735 | 24.272 | Sensitive | 30.87 | 16.113 | Sensitive | Yes |
| K. pneumoniae | Levofloxacin | 34.466 | | Sensitive | 32.085 | | Sensitive | Yes |
| K. pneumoniae | TMP/SMX | 34.918 | | Sensitive | 29.719 | | Sensitive | Yes |
| K. pneumoniae | Nitrofurantoin | 33.13 | | Sensitive | 28.708 | | Sensitive | Yes |
| K. pneumoniae | Fosfomycin | 18.711 | | Resistant | 14.895 | | Resistant | Yes |
| K. pneumoniae | Amp/Sulbactam | 23.602 | | Resistant | 15.608 | | Resistant | Yes |
| K. pneumoniae | Ampicillin | 21.189 | | Resistant | 14.269 | | Resistant | Yes |
| K. pneumoniae | Cefazolin | 32.648 | | Sensitive | 29.064 | | Sensitive | Yes |
| K. pneumoniae | Cefoxitin | 32.55 | | Sensitive | 27.384 | | Sensitive | Yes |
| K. pneumoniae | Cefepime | 32.856 | | Sensitive | 28.991 | | Sensitive | Yes |
| K. pneumoniae | Ceftriaxone | 33.145 | | Sensitive | 28.004 | | Sensitive | Yes |
| K. pneumoniae | Gentamicin | 44.195 | | Sensitive | 23.5 | | Sensitive | Yes |
| K. pneumoniae | Meropenem | 31.816 | | Sensitive | 26.606 | | Sensitive | Yes |
| K. pneumoniae | Pip/Tazo | 34.274 | | Sensitive | 28.974 | | Sensitive | Yes |
| K. pneumoniae | Tetracycline | 30.426 | | Sensitive | 27.163 | | Sensitive | Yes |
| K. aerogenes | Ciprofloxacin | 37.969 | 19.994 | Sensitive | 31.78 | 13.854 | Sensitive | Yes |
| K. aerogenes | Levofloxacin | 38.019 | | Sensitive | 33.354 | | Sensitive | Yes |
| K. aerogenes | TMP/SMX | 36.5 | | Sensitive | 30.511 | | Sensitive | Yes |
| K. aerogenes | Nitrofurantoin | 38.755 | | Sensitive | 29.821 | | Sensitive | Yes |
| K. aerogenes | Fosfomycin | 25.008 | | Sensitive | 19.581 | | Sensitive | Yes |
| K. aerogenes | Amp/Sulbactam | 31.018 | | Sensitive | 29.276 | | Sensitive | Yes |
| K. aerogenes | Ampicillin | 19.504 | | Resistant | 16.046 | | Sensitive | No |
| K. aerogenes | Cefazolin | 20.608 | | Resistant | 16.852 | | Sensitive | No |
| K. aerogenes | Cefoxitin | 20.635 | | Resistant | 13.468 | | Resistant | Yes |
| K. aerogenes | Cefepime | 32.421 | | Sensitive | 28.775 | | Sensitive | Yes |
| K. aerogenes | Ceftriaxone | 33.124 | | Sensitive | 29.137 | | Sensitive | Yes |
| K. aerogenes | Gentamicin | 36.1 | | Sensitive | 31.347 | | Sensitive | Yes |
| K. aerogenes | Meropenem | 33.144 | | Sensitive | 28.604 | | Sensitive | Yes |
| K. aerogenes | Pip/Tazo | 32.586 | | Sensitive | 30.839 | | Sensitive | Yes |
| K. aerogenes | Tetracycline | 37.04 | | Sensitive | 30.787 | | Sensitive | Yes |
| E. faecalis | Ciprofloxacin | 39.445 | 20.56 | Sensitive | 34.658 | 18.551 | Sensitive | Yes |
| E. faecalis | Levofloxacin | 37.926 | | Sensitive | 35.719 | | Sensitive | Yes |
| E. faecalis | Ampicillin | Undet | | Sensitive | 32.884 | | Sensitive | Yes |
| E. faecalis | Nitrofurantoin | Undet | | Sensitive | 37.634 | | Sensitive | Yes |
| E. faecalis | Fosfomycin | 37.968 | | Sensitive | 32.048 | | Sensitive | Yes |
| E. faecalis | Tetracycline | 23.723 | | sensitive | 21.132 | | sensitive | Yes |
| E. faecalis | Vancomycin | Undet | | Sensitive | 35.428 | | Sensitive | Yes |
| E. coli | Ciprofloxacin | 34.256 | 20.146 | Sensitive | 29.773 | 12.894 | Sensitive | Yes |
| E. coli | Levofloxacin | 34.012 | | Sensitive | 29.603 | | Sensitive | Yes |
| E. coli | TMP/SMX | 33.402 | | Sensitive | 28.739 | | Sensitive | Yes |
| E. coli | Nitrofurantoin | 34.794 | | Sensitive | 27.377 | | Sensitive | Yes |
| E. coli | Fosfomycin | 30.261 | | Sensitive | 27.932 | | Sensitive | Yes |
| E. coli | Amp/Sulbactam | 29.662 | | Sensitive | 27.583 | | Sensitive | Yes |
| E. coli | Ampicillin | 29.961 | | Sensitive | 28.274 | | Sensitive | Yes |
| E. coli | Cefazolin | 29.902 | | Sensitive | 27.921 | | Sensitive | Yes |
| E. coli | Cefoxitin | 31.631 | | Sensitive | 29.986 | | Sensitive | Yes |
| E. coli | Cefepime | 30.218 | | Sensitive | 28.12 | | Sensitive | Yes |
| E. coli | Ceftriaxone | 29.851 | | Sensitive | 30.396 | | Sensitive | Yes |
| E. coli | Gentamicin | 32.759 | | Sensitive | 28.667 | | Sensitive | Yes |
| E. coli | Meropenem | 30.723 | | Sensitive | 30.093 | | Sensitive | Yes |
| E. coli | Pip/Tazo | 30.693 | | Sensitive | 29.34 | | Sensitive | Yes |
| E. coli | Tetracycline | 34.28 | | Sensitive | 30.717 | | Sensitive | Yes |

*Undet: Undetermined, i.e. no amplification detected by PCR.

Table 7 illustrates the results of an example antimicrobial agent susceptibility test of the current application versus a traditional culture-based antimicrobial agent susceptibility test at multiple incubation timepoints, in accordance with one or more embodiments of the disclosure. These specimens were tested with direct PCR (e.g., no extraction DNA purification step prior to PCR). As in Tables 1, 2, 3, and 5, the results of the test are similar at each timepoint to the traditional test, regardless of the antibiotic exposure incubation timeline employed.

TABLE 7
Molecular phenotypic antimicrobial susceptibility results with direct PCR for various incubation timepoints.

| | | | | Antibiotic | | | Molecular | |
|---|---|---|---|---|---|---|---|---|
| | | | Traditional | Incubation | Ct at | | Antimicrobial | |
| | Isolate | | Culture | Time | Sensitive | Control | Susceptibility | |
| Pathogen | Num. | Antimicrobial | Result | (hours) | Breakpoint | Average | Result | Match? |
| E. coli | 1189 | Amoxicillin/Clavulanate | Resistant | 4 | 16.799 | 16.678 | Resistant | Yes |
| E. coli | 1189 | Ampicillin | Resistant | 4 | 16.684 | | Resistant | Yes |
| E. coli | 1189 | Cefazolin | Resistant | 4 | 16.209 | | Resistant | Yes |
| E. coli | 1189 | Cefepime | Sensitive | 4 | 20.399 | | Sensitive | Yes |
| E. coli | 1189 | Cefoxitin | Resistant | 4 | 17.798 | | Resistant | Yes |
| E. coli | 1189 | Ceftriaxone | Resistant | 4 | 16.185 | | Resistant | Yes |
| E. coli | 1189 | Ciprofloxacin | Sensitive | 4 | 27.666 | | Sensitive | Yes |
| E. coli | 1189 | Gentamicin | Sensitive | 4 | 24.224 | | Sensitive | Yes |
| E. coli | 1189 | Levofloxacin | Sensitive | 4 | 27.130 | | Sensitive | Yes |
| E. coli | 1189 | Meropenem | Sensitive | 4 | 22.751 | | Sensitive | Yes |
| E. coli | 1189 | Nitrofurantoin | Sensitive | 4 | 27.265 | | Sensitive | Yes |
| E. coli | 1189 | Piperacillin/Tazobactam | Sensitive | 4 | 20.904 | | Sensitive | Yes |
| E. coli | 1189 | TMP/SMX | Sensitive | 4 | 24.603 | | Sensitive | Yes |
| E. coli | 1189 | Amoxicillin/Clavulanate | Resistant | 6 | 17.105 | 16.992 | Resistant | Yes |
| E. coli | 1189 | Ampicillin | Resistant | 6 | 15.362 | | Resistant | Yes |
| E. coli | 1189 | Cefazolin | Resistant | 6 | 16.328 | | Resistant | Yes |
| E. coli | 1189 | Cefepime | Sensitive | 6 | 20.178 | | Sensitive | Yes |
| E. coli | 1189 | Cefoxitin | Resistant | 6 | 15.865 | | Resistant | Yes |
| E. coli | 1189 | Ceftriaxone | Resistant | 6 | 16.838 | | Resistant | Yes |
| E. coli | 1189 | Ciprofloxacin | Sensitive | 6 | 27.144 | | Sensitive | Yes |
| E. coli | 1189 | Gentamicin | Sensitive | 6 | 25.270 | | Sensitive | Yes |
| E. coli | 1189 | Levofloxacin | Sensitive | 6 | 26.759 | | Sensitive | Yes |
| E. coli | 1189 | Meropenem | Sensitive | 6 | 22.884 | | Sensitive | Yes |
| E. coli | 1189 | Nitrofurantoin | Sensitive | 6 | 27.430 | | Sensitive | Yes |
| E. coli | 1189 | Piperacillin/Tazobactam | Sensitive | 6 | 21.624 | | Sensitive | Yes |
| E. coli | 1189 | TMP/SMX | Sensitive | 6 | 23.984 | | Sensitive | Yes |
| E. coli | 1189 | Amoxicillin/Clavulanate | Resistant | 8 | 17.430 | 17.336 | Resistant | Yes |
| E. coli | 1189 | Ampicillin | Resistant | 8 | 17.281 | | Resistant | Yes |
| E. coli | 1189 | Cefazolin | Resistant | 8 | 17.305 | | Resistant | Yes |
| E. coli | 1189 | Cefepime | Sensitive | 8 | 21.204 | | Sensitive | Yes |
| E. coli | 1189 | Cefoxitin | Resistant | 8 | 16.837 | | Resistant | Yes |
| E. coli | 1189 | Ceftriaxone | Resistant | 8 | 17.431 | | Resistant | Yes |
| E. coli | 1189 | Ciprofloxacin | Sensitive | 8 | 28.104 | | Sensitive | Yes |
| E. coli | 1189 | Gentamicin | Sensitive | 8 | 25.454 | | Sensitive | Yes |
| E. coli | 1189 | Levofloxacin | Sensitive | 8 | 27.068 | | Sensitive | Yes |
| E. coli | 1189 | Meropenem | Sensitive | 8 | 23.187 | | Sensitive | Yes |
| E. coli | 1189 | Nitrofurantoin | Sensitive | 8 | 28.823 | | Sensitive | Yes |
| E. coli | 1189 | Piperacillin/Tazobactam | Sensitive | 8 | 21.570 | | Sensitive | Yes |
| E. coli | 1189 | TMP/SMX | Sensitive | 8 | 24.482 | | Sensitive | Yes |
| E. coli | 1189 | Amoxicillin/Clavulanate | Resistant | 10 | 15.606 | 15.995 | Resistant | Yes |
| E. coli | 1189 | Ampicillin | Resistant | 10 | 16.280 | | Resistant | Yes |
| E. coli | 1189 | Cefazolin | Resistant | 10 | 15.756 | | Resistant | Yes |
| E. coli | 1189 | Cefepime | Sensitive | 10 | 21.613 | | Sensitive | Yes |
| E. coli | 1189 | Cefoxitin | Resistant | 10 | 14.899 | | Resistant | Yes |
| E. coli | 1189 | Ceftriaxone | Resistant | 10 | 16.347 | | Resistant | Yes |
| E. coli | 1189 | Ciprofloxacin | Sensitive | 10 | 27.715 | | Sensitive | Yes |
| E. coli | 1189 | Gentamicin | Sensitive | 10 | 25.318 | | Sensitive | Yes |
| E. coli | 1189 | Levofloxacin | Sensitive | 10 | 27.170 | | Sensitive | Yes |
| E. coli | 1189 | Meropenem | Sensitive | 10 | 23.901 | | Sensitive | Yes |
| E. coli | 1189 | Nitrofurantoin | Sensitive | 10 | 27.170 | | Sensitive | Yes |
| E. coli | 1189 | Piperacillin/Tazobactam | Sensitive | 10 | 22.310 | | Sensitive | Yes |
| E. coli | 1189 | TMP/SMX | Sensitive | 10 | 24.129 | | Sensitive | Yes |
| E. coli | 1189 | Amoxicillin/Clavulinate | Resistant | 12 | 16.125 | 15.495 | Resistant | Yes |
| E. coli | 1189 | Ampicillin | Resistant | 12 | 15.183 | | Resistant | Yes |
| E. coli | 1189 | Cefazolin | Resistant | 12 | 15.126 | | Resistant | Yes |
| E. coli | 1189 | Cefepime | Sensitive | 12 | 22.447 | | Sensitive | Yes |
| E. coli | 1189 | Cefoxitin | Resistant | 12 | 15.750 | | Resistant | Yes |
| E. coli | 1189 | Ceftriaxone | Resistant | 12 | 16.258 | | Resistant | Yes |
| E. coli | 1189 | Ciprofloxacin | Sensitive | 12 | 27.182 | | Sensitive | Yes |
| E. coli | 1189 | Gentamicin | Sensitive | 12 | 25.018 | | Sensitive | Yes |
| E. coli | 1189 | Levofloxacin | Sensitive | 12 | 26.835 | | Sensitive | Yes |
| E. coli | 1189 | Meropenem | Sensitive | 12 | 26.014 | | Sensitive | Yes |
| E. coli | 1189 | Nitrofurantoin | Sensitive | 12 | 26.777 | | Sensitive | Yes |
| E. coli | 1189 | Piperacillin/Tazobactam | Sensitive | 12 | 22.359 | | Sensitive | Yes |
| E. coli | 1189 | TMP/SMX | Sensitive | 12 | 23.878 | | Sensitive | Yes |
| E. coli | 1189 | Amoxicillin/Clavulanate | Resistant | 14 | 14.226 | 15.826 | Resistant | Yes |
| E. coli | 1189 | Ampicillin | Resistant | 14 | 15.465 | | Resistant | Yes |
| E. coli | 1189 | Cefazolin | Resistant | 14 | 17.599 | | Resistant | Yes |
| E. coli | 1189 | Cefepime | Sensitive | 14 | 22.420 | | Sensitive | Yes |
| E. coli | 1189 | Cefoxitin | Resistant | 14 | 15.333 | | Resistant | Yes |
| E. coli | 1189 | Ceftriaxone | Resistant | 14 | 14.724 | | Resistant | Yes |
| E. coli | 1189 | Ciprofloxacin | Sensitive | 14 | 27.128 | | Sensitive | Yes |
| E. coli | 1189 | Gentamicin | Sensitive | 14 | 24.069 | | Sensitive | Yes |
| E. coli | 1189 | Levofloxacin | Sensitive | 14 | 27.545 | | Sensitive | Yes |
| E. coli | 1189 | Meropenem | Sensitive | 14 | 26.211 | | Sensitive | Yes |
| E. coli | 1189 | Nitrofurantoin | Sensitive | 14 | 26.788 | | Sensitive | Yes |
| E. coli | 1189 | Piperacillin/Tazobactam | Sensitive | 14 | 24.257 | | Sensitive | Yes |
| E. coli | 1189 | TMP/SMX | Sensitive | 14 | 24.017 | | Sensitive | Yes |
| E. coli | 1189 | Amoxicillin/Clavulanate | Resistant | 16 | 14.825 | 14.880 | Resistant | Yes |
| E. coli | 1189 | Ampicillin | Resistant | 16 | 15.036 | | Resistant | Yes |
| E. coli | 1189 | Cefazolin | Resistant | 16 | 15.199 | | Resistant | Yes |
| E. coli | 1189 | Cefepime | Sensitive | 16 | 23.502 | | Sensitive | Yes |
| E. coli | 1189 | Cefoxitin | Resistant | 16 | 13.704 | | Resistant | Yes |
| E. coli | 1189 | Ceftriaxone | Resistant | 16 | 15.840 | | Resistant | Yes |
| E. coli | 1189 | Ciprofloxacin | Sensitive | 16 | 28.446 | | Sensitive | Yes |
| E. coli | 1189 | Gentamicin | Sensitive | 16 | 24.816 | | Sensitive | Yes |
| E. coli | 1189 | Levofloxacin | Sensitive | 16 | 27.791 | | Sensitive | Yes |
| E. coli | 1189 | Meropenem | Sensitive | 16 | 27.457 | | Sensitive | Yes |
| E. coli | 1189 | Nitrofurantoin | Sensitive | 16 | 24.548 | | Sensitive | Yes |
| E. coli | 1189 | Piperacillin/Tazobactam | Sensitive | 16 | 24.143 | | Sensitive | Yes |
| E. coli | 1189 | TMP/SMX | Sensitive | 16 | 23.552 | | Sensitive | Yes |

Table 8 illustrates the results of an example antimicrobial agent susceptibility test of the current application versus a traditional culture-based antimicrobial agent susceptibility test for a low level positive with <10,000 cells/mL in the original specimen. These specimens were tested with direct PCR (e.g., no extraction DNA purification step prior to PCR) after 16 hours of incubation. As in Tables 1, 2, 3, 5, and 7, the results of the test are similar to the traditional test.

TABLE 8
Molecular phenotypic antimicrobial susceptibility results with direct PCR for specimen with <10,000 cells/mL.

| | | | | | | | Molecular | |
|---|---|---|---|---|---|---|---|---|
| | | Ct of | | Traditional | Ct at | | Antimicrobial | |
| | | original | | Culture | Sensitive | Ct at | Susceptibility | |
| Pathogen | Isolate number | specimen | Antimicrobial | Results | Breakpoint | Control | Result | Match? |
| E. coli | #1 - WDCM 00090 - 6.2×E3 | 27.277 | Nitrofurantoin | Sensitive | 27.90 | 13.787 | Sensitive | Yes |
| E. coli | #1 - WDCM 00090 - 6.2×E3 | 27.277 | Fosfomycin | Sensitive | 27.59 | | Sensitive | Yes |
| E. coli | #1 - WDCM 00090 - 6.2×E3 | 27.277 | Ampicillin | Sensitive | 25.93 | | Sensitive | Yes |
| E. coli | #1 - WDCM 00090 - 6.2×E3 | 27.277 | Cefazolin | Sensitive | 25.72 | | Sensitive | Yes |
| E. coli | #1 - WDCM 00090 - 6.2×E3 | 27.277 | Ceftriaxone | Sensitive | 26.31 | | Sensitive | Yes |
| E. coli | #1 - WDCM 00090 - 6.2×E3 | 27.277 | Gentamicin | Sensitive | 28.57 | | Sensitive | Yes |
| E. coli | #1 - WDCM 00090 - 6.2×E3 | 27.277 | Piperacillin/Tazobactam | Sensitive | 26.78 | | Sensitive | Yes |
| E. coli | #1 - WDCM 00090 - 6.2×E3 | 27.277 | Doxycycline | Sensitive | 25.83 | | Sensitive | Yes |
| E. coli | #2 - WDCM 00090 - 6.2×E3 | 27.277 | Nitrofurantoin | Sensitive | 28.97 | 13.46 | Sensitive | Yes |
| E. coli | #2 - WDCM 00090 - 6.2×E3 | 27.277 | Fosfomycin | Sensitive | 28.41 | | Sensitive | Yes |
| E. coli | #2 - WDCM 00090 - 6.2×E3 | 27.277 | Ampicillin | Sensitive | 26.22 | | Sensitive | Yes |
| E. coli | #2 - WDCM 00090 - 6.2×E3 | 27.277 | Cefazolin | Sensitive | 26.56 | | Sensitive | Yes |
| E. coli | #2 - WDCM 00090 - 6.2×E3 | 27.277 | Ceftriaxone | Sensitive | 27.18 | | Sensitive | Yes |
| E. coli | #2 - WDCM 00090 - 6.2×E3 | 27.277 | Gentamicin | Sensitive | 28.70 | | Sensitive | Yes |
| E. coli | #2 - WDCM 00090 - 6.2×E3 | 27.277 | Piperacillin/Tazobactam | Sensitive | 27.46 | | Sensitive | Yes |
| E. coli | #2 - WDCM 00090 - 6.2×E3 | 27.277 | Doxycycline | Sensitive | 27.02 | | Sensitive | Yes |

Table 9 illustrates an antimicrobial exposure plate for Enterobacterales organisms with specified antimicrobial agents at known concentrations (μg/mL), in accordance with one or more embodiments of the disclosure.

TABLE 9
Antimicrobial exposure plate for Enterobacterales organisms
with specified antimicrobials contents at known concentrations

| | | Antimicrobial |
|---|---|---|
| Well | | Concentration |
| Location | Antimicrobial Agent | (μg/mL) |
| A1 | None - Control | 0 |
| B1 | None - Control | 0 |
| C1 | None - Control | 0 |
| D1 | Ciprofloxacin | 0.25 |
| E1 | Levofloxacin | 0.5 |
| F1 | Trimethoprim/Sulfamethoxazole | 2/38 |
| G1 | Nitrofurantoin | 32 |
| H1 | Fosfomycin/Glucose 6 phosphate | 64/25 |
| A2 | Ampicillin/Sulbactam | 8/4 |
| B2 | Ampicillin | 8 |
| C2 | Cefazolin | 2 |
| D2 | Cefoxitin | 8 |
| E2 | Cefepime | 2 |
| F2 | Ceftriaxone | 1 |
| G2 | Gentamicin | 2 |
| H2 | Meropenem | 1 |
| A2 | Piperacillin/Tazobactam | 8/4 |
| B2 | Doxycycline | 4 |

Table 10 illustrates an antimicrobial exposure plate for Enterococcus organisms with specified antimicrobial agents at known concentrations (μg/mL), in accordance with one or more embodiments of the disclosure.

TABLE 10
Antimicrobial exposure plate for Enterococcus species organisms
with specified antimicrobials contents at known concentrations

| | | Antimicrobial |
|---|---|---|
| Well | | Concentration |
| Location | Antimicrobial Agent | (μg/mL) |
| A1 | None - Control | 0 |
| B1 | None - Control | 0 |
| C1 | None - Control | 0 |
| D1 | Ciprofloxacin | 1 |
| E1 | Levofloxacin | 2 |
| F1 | Ampicillin | 8 |
| G1 | Nitrofurantoin | 32 |
| H1 | Fosfomycin/Glucose 6 phosphate | 64/25 |
| A2 | Doxycycline | 4 |
| B2 | Vancomycin | 4 |

Table 11 illustrates an antimicrobial exposure plate for Pseudomonas aeruginosa organism with specified antimicrobial agents at known concentrations (μg/mL), in accordance with one or more embodiments of the disclosure.

TABLE 11
Antimicrobial exposure plate for Pseudomonas aeruginosa species
organisms with specified contents at known concentrations

| | | Antimicrobial |
|---|---|---|
| Well | | Concentration |
| Location | Antimicrobial Agent | (μg/mL) |
| A1 | None - Control | 0 |
| B1 | None - Control | 0 |
| C1 | None - Control | 0 |
| D1 | Ciprofloxacin | 0.5 |
| E1 | Levofloxacin | 1 |
| F1 | Meropenem | 2 |
| G1 | Piperacillin/Tazobactam | 16/4 |
| H1 | Ceftazidime | 8 |
| A2 | Cefepime | 8 |

Table 12 illustrates an antimicrobial exposure plate for Acinetobacter baumannii organism with specified antimicrobial agents at known concentrations (μg/mL), in accordance with one or more embodiments of the disclosure.

TABLE 12
Antimicrobial exposure plate for Acinetobacter baumannii organism
with specified antimicrobials contents at known concentrations

| | | Antimicrobial |
|---|---|---|
| Well | | Concentration |
| Location | Antimicrobial Agent | (μg/mL) |
| A1 | None - Control | 0 |
| B1 | None - Control | 0 |
| C1 | None - Control | 0 |
| D1 | Ciprofloxacin | 1 |
| E1 | Levofloxacin | 2 |
| F1 | Trimethoprim/Sulfamethoxazole | 2/38 |
| G1 | Piperacillin/Tazobactam | 16/4 |
| H1 | Doxycycline | 4 |
| A2 | Ampicillin/Sulbactam | 8/4 |
| B2 | Meropenem | 2 |
| C2 | Gentamicin | 4 |
| D2 | Ceftriaxone | 8 |
| E2 | Cefepime | 8 |

Table 13 illustrates an antimicrobial exposure plate for Staphylococcus aureus organism with specified antimicrobial agents at known concentrations (μg/mL), in accordance with one or more embodiments of the disclosure.

TABLE 13
Antimicrobial exposure plate for Staphylococcus aureus organism
with specified antimicrobials contents at known concentrations

| | | Antimicrobial |
|---|---|---|
| Well | | Concentration |
| Location | Antimicrobial Agent | (μg/mL) |
| A1 | None - Control | 0 |
| B1 | None - Control | 0 |
| C1 | None - Control | 0 |
| D1 | Ciprofloxacin | 1 |
| E1 | Levofloxacin | 1 |
| F1 | Trimethoprim/Sulfamethoxazole | 2/38 |
| G1 | Nitrofurantoin | 32 |
| H1 | Gentamicin | 4 |
| A2 | Doxycycline | 4 |
| B2 | Vancomycin | 2 |
| C2 | Cefoxitin | 4 |

### Example 2. Development of a Panel for Pathogen Identification

Identification of Pathogen of Interest and DNA Targets. A panel of specific pathogens was created based on the specimen source and organisms known to cause infection in that source. To maximize the number of pathogens able to be tested with the least number of specimen wells, a multi-plex PCR method is utilized. Different fluorescent probes are utilized for each pathogen in a single well. Table 12A illustrates a first set of pathogens of interest, as well as genetic targets for identification of several of the pathogens of interest via the AST of the current application. Table 12B illustrates a second set of pathogens of interest, as well as genetic targets for identification of several of the pathogens of interest via the AST of the current application. The AST may be configured to detect one or more pathogens from tables 14A-14B.

TABLE 14A
Pathogens of interest and genetic target for identification

| Pathogen Name | Target |
|---|---|
| Acinetobacter baumannii | OmpA family protein - CP053098.1: 902573-903226 |
| Candida albicans | agglutinin-like protein 1 (ALS1) gene |
| Candida glabrata | Pdr1 (PDR1) gene |
| Candida parapsilosis | MET2 gene |
| Citrobacter freundii | cyclopropane fatty acid synthase (cfa) gene, dihydroxyacetone |
| | kinase (dhaK), glycerol dehydrogenase (dhaD), transcriptional |
| | activator (dhaR), glycerol dehydratase reactivation factor |
| | small subunit (dhaG), 1,3-propanediol dehydrogenase (dhaT), |
| | glycerol dehydratase 60 kDa subunit (dhaB), glycerol dehydratase |
| | 21 kDa subunit (dhaC), glycerol dehydratase 16 kDa subunit |
| | (dhaE), and glycerol dehydratase reactivation factor large |
| | subunit (dhaF) genes |
| Enterobacter aerogenes | outer membrane porin protein (omp35) gene |
| Enterobacter cloacae | Hsp60 (hsp60) gene |
| Enterococcus faecalis | D-alanine ligase-related protein (ddl) gene |
| Enterococcus faecium | |
| Escherichia coli | universal stress protein UspB |
| Klebsiella oxytoca | gyrase A (gyrA) gene |
| Klebsiella pneumoniae | NC_012731.1 |
| Morganella morgani | 16S ribosomal RNA gene |
| Mycoplasma hominis | CP055149.1 |
| Proteus mirablis | ureR gene |
| Proteus vulgaris | gyrase beta-subunit (gyrB) gene |
| Providencia stuartii | CP008920.1 |
| Pseudomonas aeruginosa | exotoxin A precursor (toxA) gene |
| Rnase P | Homo sapiens ribonuclease P RNA component H1 (RPPH1) |
| Serratia marcescens | AP021873.1 |
| Staphylococcus aureus | thermonuclease gene |
| Staphylococcus saprophyticus | Tuf (tuf) gene |
| Streptococcus agalactiae | CP053027.1 |
| Ureaplasma urealyticum | urease complex component (ureG) gene |

TABLE 14B
Future organisms
of interest

| | |
|---|---|
| Actinotignum schaalii | |
| Aerococcus urinae | |
| Bacteroides fragilis | |
| Candida auris | |
| Chlamydia trachomatis | outer membrane protein (ompA) gene |
| Citrobacter koseri | |
| Corynebacterium riegelii | |
| Gardnerella vaginalis | vaginolysin (vly) gene |
| Mycoplasma genatalium | BLG302 adhesin protein (mgpB) gene |
| Neisseria gonorrhoeae | opak gene for opacity protein |
| Pantoea agglomerans | |
| Staphylococcus epidermidis | |
| Staphylococcus saprophyticus | |
| Trichomonas vaginalis | 18S ribosomal RNA gene |

### Example 3. Evaluation of Antimicrobial Resistance Genes

Table 15 illustrates a list of evaluated antimicrobial resistance genes, in accordance with one or more embodiments of the disclosure.

TABLE 15

| Resistance | |
|---|---|
| Gene | Target |
| AmpC | blaACT gene for cephalosporin-hydrolyzing class |
| | C beta-lactamase ACT-79 |
| FemA | methicillin resistance (femA) gene and trpA gene |
| MECA | mecA gene for penicillin binding protein 2a |
| QNRA | Quinolone resistance pentapeptide repeat protein (gnrA) gene |
| QNRB | quinolone resistance pentapeptide repeat protein (gnrB) gene |
| VANA1 | vancomycin A-type 1 resistance protein (vanA) gene |
| VANA2 | vancomycin A-type 2 resistance protein (vanA) gene |
| VANB | vancomycin resistance protein B (vanB) gene |
| KPC | class A beta-lactamase KPC-59 (blaKPC) gene |
| NDM | metallo-beta-lactamase NDM-5 (blaNDM) gene |
| OXA-48 | beta-lactamase OXA-920 (blaOXA) gene |
| IMP-7 | blaVIM-1 gene for metallo-beta-lactamase VIM-1 |
| VIM | blaVIM-1 gene for metallo-beta-lactamase VIM-1 |
| CTXM1 | bla(CTX-M-1) gene |
| CTXM2 | beta-lactamase CTX-M-2 (blaCTX-M) gene |
| SHV | broad-spectrum beta-lactamase SHV-11 (blaSHV) gene |
| TEM | plasmid pRHB16-C07_2 |
| SUL2 | Sulfamethoxzole |
| | Trimethoprim |
| ErmA | Erythromycin |
| ErmB | Erythromycin |
| ErmC | Erythromycin |

### Example 4. Reporting AST Results

Result Reporting to Healthcare Providers. The results from the antimicrobial exposure are reported to the ordering healthcare provider. This information can be utilized by the healthcare provider when selecting an antimicrobial regimen for their individual patient based on these individual specimen results and patient specific factors. The healthcare provider could initiate a new antimicrobial regimen or change an existing regimen based on these results. The current standard of care for antimicrobial susceptibility testing involves the growth, isolation, and exposure of pathogens to antimicrobials. This traditional culture and sensitivity process can take multiple days to obtain results, limiting the ability of healthcare providers to receive timely results to make actionable changes in patient care or management. Healthcare providers desire a method to obtain antimicrobial susceptibility results with quicker turn-around-time. This application discloses a molecular-based phenotypic antimicrobial susceptibility method which involves pathogen exposure to a panel of antimicrobial agents at their specified minimum inhibitory concentration breakpoints for sensitivity. This method allows for more timely antimicrobial susceptibility results.

As detailed above, the specimen 100 may be divided into more than two samples. Also, although the first sample 104 is illustrated as being used for pathogen identification, and the second sample 108 is illustrated as being used for antimicrobial agent susceptibility, the specimen 100, the first sample 104, the second sample 108, or any other sample taken from the specimen 100 may be used for any step of the methods described herein.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

Although particular embodiments of this invention have been illustrated, it is apparent that various modifications and embodiments of the invention may be made by those skilled in the art without departing from the scope and spirit of the foregoing disclosure. It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Accordingly, the scope of the invention should be limited only by the claims appended hereto.

As used herein, “amplification” refers to the process of semi-conservatively replicating nucleic acid strands by enzyme-catalyzed extension. Exemplary enzymes for amplification of nucleic acids in the current disclosure include, for example, nucleic acid polymerases. In some embodiments, an isothermal polymerase is used to amplify nucleic acids. In some embodiments, amplification is carried out with a high-fidelity polymerase with the technique known as the polymerase chain reaction (PCR). Amplification can be performed with natural and nonnatural nucleotide bases, ribonucleotide bases or deoxyribonucleotide bases, labeled nucleotide bases, and the like.

As detailed herein, obtaining a sample and performing PCR may also include harvesting the pathogen and isolating pathogen DNA from the harvested pathogens for amplification by PCR. Harvesting the pathogen may be performed by methods (e.g., centrifugation, kits) that are well-known to the skilled artisan. Isolating pathogen DNA from the harvested pathogens may performed by methods (e.g., phenol extraction/kits) that are well-known to the skilled artisan.

As used herein, the norm “well” refers to a single container or reaction vessel. Though the term well is often used when referring to plates or microplates, it is to be understood that the methods of the current disclosure may also be performed using, for example, tubes or other vessels capable of containing and separating liquids.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O (6)-methylguanine, and2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having six members refers to groups having 1, 2, 3, 4, or 6 members, and so forth.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For purposes of this disclosure, “treating” or “treatment” describes the management and care of a patient for the purpose of combating the disease, condition, or disorder. The terms embrace both preventative, i.e., prophylactic, and palliative treatment. “Treating” includes the administration of a composition of present disclosure to prevent the onset of the symptoms or complications, alleviating the symptoms or complications, or eliminating the disease, condition, or disorder. The term “treat” and words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of this disclosure can provide any amount of any level of treatment or prevention of disease in a mammal. Furthermore, the treatment or prevention provided by the inventive method can include treatment or prevention of one or more conditions or symptoms of the disease or disease state. Also, for purposes herein, “prevention” can encompass delaying the onset of the disease, or a symptom or condition thereof for purposes of the present disclosure, “treating” or “treatment” comprises the management and care of a subject for the purpose of combating a disease, condition, or disorder. Treating includes the administration of a probiotic and oxytocin as described herein to prevent the onset of the symptoms or complications, and/or to alleviate the symptoms or complications of a disease, condition, or disorder.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the duration of the treatment, the nature of concurrent therapy (if any), and the specific formulations employed and the structure of the therapeutic or its derivatives. The exact dosage is chosen by the individual physician in view of the patient to be treated. Dosage and administration are adjusted to provide sufficient levels of the active agent(s) or to maintain the desired effect.

In some embodiments, the optimum effective amounts can be readily determined by one of ordinary skill in the art using routine experimentation. In some embodiments, a therapeutically effective amount is achieved by administering multiple therapeutically effective doses, e.g., over the course of a day, several days, a week, several weeks, months, or years. In some embodiments, an effective amount is administered as symptoms dictate.

What is claimed:

1. A method for identifying antimicrobial susceptibility of one or more microorganisms in a biological specimen of a subject comprising:
obtaining the biological specimen;
dividing the specimen into at least a first sample and a second sample;
identifying at least one pathogen in the first sample via genetic analysis;
exposing the second sample to a panel of antimicrobial agents at known concentrations based on the at least one pathogen;
performing a polymerase chain reaction (PCR) assay on the second sample after exposure of the second sample to the panel of the antimicrobial agents, wherein the PCR assay includes oligonucleotides that amplify a nucleic acid sequence for one or more of the identified pathogens; and
analyzing a result of the PCR assay to determine one or more antimicrobial agents that at least one of:
inhibit a growth of one or more species of pathogens;
reduce a presence of the one or more species of the pathogens; or
allow for a growth of the one or more species of the pathogens.

2. The method of claim 1, further comprising preparing the first sample for the genetic analysis.

3. The method of claim 2, wherein preparing the first sample for the genetic analysis comprises:
adding a lysis buffer to the first sample; and
adding a portion of the lysis buffer to a PCR reaction.

4. The method of claim 2, wherein preparing the first sample for the genetic analysis comprises:
isolating genetic material from the first sample; and
adding a portion of the genetic material to a PCR reaction.

5. The method of claim 1, further comprising, before performing the PCR assay on the second sample, incubating the second sample in a liquid culture media.

6. The method of claim 1, wherein the PCR assay is performed based upon an indication that at least of the specimen, the first sample, or the second sample, comprises an identified pathogen.

7. The method of claim 1, further comprising performing a PCR assay configured to amplify a portion of one or more drug-resistance genes associated with the pathogen from at least one of the biological specimen, the first sample, or the second sample.

8. The method of claim 1, further comprising reporting a result of an analysis of the result of the PCR assay to a medical provider.

9. The method of claim 8, further comprising administering an effective amount of an antimicrobial agent to the subject based on the analysis of the result of the PCR assay.

10. The method of claim 1, wherein identifying the at least one pathogen in the first sample via the genetic analysis comprises performing a multiplex PCR reaction.

11. The method of claim 10, wherein the multiplex PCR reaction is configured to amplify an identifying DNA sequence from an Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.

12. The method of claim 11 wherein the multiplex PCR is further configured to amplify an identifying sequence from Morganella morganii, Ureaplasma urealyticum, and Enterobacter cloacae.

13. The method of claim 1, wherein the PCR assay comprises semi-quantitative PCR.

14. The method of claim 1, wherein the biological specimen comprises at least one of urine, blood, saliva, feces, pleural fluid, mucosal samples, or cerebral spinal fluid.

15. The method of claim 7, wherein the panel of the antimicrobial agents comprises ciprofloxacin and levofloxacin.

16. A kit comprising a polymerase chain reaction (PCR) master mix, wherein the PCR master mix comprises:
a set of primers for amplifying an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.;
a polymerase enzyme; and
a PCR buffer solution.

17. The kit of claim 16, wherein the set of primers further comprises a set of primers for amplifying an identifying DNA sequence from Morganella morganii, Ureaplasma urealyticum, and Enterobacter cloacae.

18. A system comprising:
a polymerase chain reaction (PCR) master mix, wherein the PCR master mix comprises:
a set of primers for amplifying an identifying DNA sequence from Acinetobacter sp., a Candida sp., a Citrobacter sp., an Enterobacter sp., an Enterococcus sp. Escherichia sp., a Mycoplasma sp., a Proteus sp., a Pseudomonas sp., and a Staphylococcus sp.;
a polymerase enzyme; and
a PCR buffer solution; and
a set of antibiotics configured to treat one or more pathogens identified via amplification of a subject sample via the PCR master mix.

19. The system of claim 18, wherein the set of primers further comprises primers for amplifying an identifying DNA sequence from Morganella morganii, Ureaplasma urealyticum, and Enterobacter cloacae.

20. The system of claim 18, wherein the set of antibiotics comprises ciprofloxacin and levofloxacin.
